(19) United States
(12) Patent Application Publication
(10) Pub. No.: US 2025/0311706 A1
(43) Pub. Date: Oct. 9, 2025

(54) ANIMAL MODELS AND METHODS OF USE

(71) Applicant: Genentech, Inc., South San Francisco, CA (US)

(72) Inventors: Xiaowu GU, Foster City, CA (US); Tom TRUONG, San Jose, CA (US); Shawnta Yvonne CHANEY, Brisbane, CA (US); Marion Aline JEANNE, San Francisco, CA (US)

(73) Assignee: Genentech, Inc., South San Francisco, CA (US)

(21) Appl. No.: 19/242,337

(22) Filed: Jun. 18, 2025

Related U.S. Application Data
(63) Continuation of application No. PCT/US2023/084750, filed on Dec. 19, 2023.
(60) Provisional application No. 63/434,312, filed on Dec. 21, 2022.

(51) Int. Cl. A01K 67/0276; G01N 33/50

(52) U.S. Cl. CPC A01K 67/0276; G01N 33/5029; G01N 33/5064; A01K 2227/105; A01K 2227/106; A01K 2267/0331; G01N 2500/10

(57) ABSTRACT

The present disclosure provides animal models of wound healing and diseases associated with angiogenesis, such as age-related macular degeneration (AMD), fibrosis and cancer. The present disclosure further provides methods for identifying agents for promoting wound healing, modulating angiogenesis or treating diseases associated with angiogenesis, such as AMD and cancer.

### CROSS REFERENCE TO RELATED APPLICATIONS

This application is a continuation of International Patent Application No. PCT/US2023/084750, filed Dec. 19, 2023, which claims priority to U.S. Provisional Application No. 63/434,312, filed Dec. 21, 2022, the contents of which are incorporated herein by reference in their entirety.

### SEQUENCE LISTING

This application contains a Sequence Listing which has been submitted electronically in XML format and is hereby incorporated by reference in its entirety. Said XML copy, created on Jun. 4, 2025, is named P37344-US-1_SeqListing.xml and is 11,918 bytes in size.

### TECHNICAL FIELD

The present disclosure relates to animal models of modeling wound healing and diseases associated with angiogenesis, such as age-related macular degeneration (AMD), fibrosis and cancer, and methods of identifying agents for promoting wound healing, modulating angiogenesis or treating diseases associated with angiogenesis, such as AMD and cancer.

### BACKGROUND

Age-related macular degeneration (AMD) is the leading cause of blindness in the elderly worldwide. AMD is a chronic and progressive degenerative disorder of the macula characterized by the loss of central vision as a result of abnormalities in the photoreceptors, retinal pigment epithelium, Bruch's membrane and choroidal complex of the eye. AMD is subdivided into early, intermediate, and late stages. Late-stage AMD is further subdivided into the dry AMD form, called Geographic Atrophy (GA), and the wet AMD form, also called neovascular AMD. Wet AMD is characterized by aberrant growth and leakage of blood vessels in the retina. Although dry AMD accounts for the majority of all diagnosed cases, wet AMD is responsible for the majority of cases with severe vision loss, and it usually occurs over weeks to months. Although neovascularization has been the most common cause of severe vision loss, GA can cause a significant loss of vision as well. There is currently no approved treatment for intermediate AMD and GA. While wet AMD is currently treated using intravitreal injection of VEGF inhibitors, less than 40% of patients show complete response to these treatments. By contrast, existing mouse models of AMD are much more responsive to VEGF inhibitors, making it difficult to screen for and study therapies that can treat AMD that is non-responsive to VEGF inhibitors in these models. Accordingly, there is a need for novel animal models of AMD and other diseases and injuries associated with angiogenesis, as well as new methods for identifying agents that treat AMD and other diseases and injuries associated with angiogenesis.

### SUMMARY OF THE DISCLOSURE

In a first aspect, the present disclosure provides a method for determining the efficacy of an agent in modulating angiogenesis in a tissue. In some embodiments, the method comprises the steps of: (a) inducing neovascularization in the tissue of a control subject and a test subject, wherein the Collagen Type VIII Alpha 1 Chain (Col8a1) gene has been knocked out or expression of the Col8a1 gene has been knocked down in both the control and test subjects, (b) administering the agent to the test subject, and (c) measuring the size of one or more lesions resulting from the neovascularization in the control and test subjects, wherein the agent modulates angiogenesis in the tissue if the size of the one or more lesions in the test subject is smaller than the size of the one or more lesions in the control subject.

In some embodiments, the tissue is retina. In some embodiments, the neovascularization is induced by application of a laser to the basement membrane of an eye of the control subject and the test subject. In some embodiments, the tissue is a tumor. In some embodiments, the neovascularization is induced by transplantation of a cell matrix plug.

In some embodiments, the agent is administered to the test subject after neovascularization is induced. In some embodiments, the agent is administered to the test subject before neovascularization is induced. In some embodiments, the agent is administered to the test subject during the induction of the neovascularization.

In a second aspect, the present disclosure provides a method for determining the efficacy of an agent in promoting wound healing of an injured tissue in a subject. In some embodiments, the present disclosure provides a method for determining the efficacy of an agent in inhibiting fibrosis in an injured tissue in a subject. In some embodiments the method comprises the steps of: (a) inducing one or more lesions by injuring the tissue of a control subject and a test subject, wherein the Collagen Type VIII Alpha 1 Chain (Col8a1) gene has been knocked out or expression of the Col8a1 gene has been knocked down in both the control and test subjects, (b) administering the agent to the test subject, and (c) measuring the size of the one or more lesions and/or one or more fibrotic scars formed at the one or more lesions in the control and test subjects, wherein the agent promotes wound healing in the injured tissue if the size of the one or more lesions in the test subject is smaller than the size of the one or more lesions in the control subject, and/or if the size of the one or more fibrotic scars in the test subject is smaller than the size of the one or more fibrotic scars in the test subject; and wherein the agent inhibits fibrosis of the injured tissue if the size of the one or more fibrotic scars in the test subject is smaller than the size of the one or more fibrotic scars in the test subject.

In some embodiments, the injured tissue may be skin or kidney. In some embodiments, the injured tissue is skin. In some embodiments, the injured tissue is kidney. In some embodiments, the tissue injury is caused by surgical incision or skin biopsy punch. In some embodiments, the tissue injury is caused by surgical incision. In some embodiments, the tissue injury is induced by skin biopsy punch. In some embodiments, the injured tissue is retina.

In some embodiments, the agent is administered to the test subject after the one or more lesions are induced. In some embodiments, the agent is administered to the test subject before the one or more lesions are induced. In some embodiments, the agent is administered to the test subject during the induction of the one or more lesions.

In a third aspect, the present disclosure provides a method for determining the efficacy of an agent in treating or preventing age-related macular degeneration (AMD) in a subject. In some embodiments, the method comprises the steps of: (a) inducing one or more lesions in retina of a control subject and a test subject, wherein the Collagen Type VIII Alpha 1 Chain (Col8a1) gene has been knocked out or expression of the Col8a1 gene has been knocked down in both the control and test subjects, (b) administering the agent to the test subject, and (c) measuring the size of the one or more lesions in the control and test subjects, wherein the agent treats or prevents AMD if the size of the one or more lesions in the test subject is smaller than the size of the one or more lesions in the control subject.

In some embodiments, the AMD is neovascular AMD. In some embodiments, the one or more lesions is induced by application of a laser to the basement membrane of an eye of the control subject and the test subject. In some embodiments, the application of the laser causes choroidal neovascularization.

In some embodiments, the AMD is geographic atrophy (GA). In some embodiments, the one or more lesions is induced by injection of sodium iodate into the control subject and the test subject. In some embodiments, the sodium iodate is administered locally. In some embodiments, the sodium iodate is administered intravitreally or retro-orbitally. In some embodiments, the sodium iodate is administered systemically. In some embodiments, the sodium iodate is administered intravenously.

In some embodiments, the agent is administered to the test subject after the one or more lesions are induced. In some embodiments, the agent is administered to the test subject before the one or more lesions are induced. In some embodiments, the agent is administered to the test subject during the induction of the one or more lesions.

In some embodiments, the lesion size is measured between 5 and 10 days after the one or more lesions are induced. The lesion size may be measured 7 days after the one or more lesions are induced.

In some embodiments of any of the above aspects, the size of the one or more lesions or fibrotic scars is measured by fluorescent, histological and/or optical coherence tomography analysis, such as by fluorescent microscopy.

In a fourth aspect, the present disclosure provides a method for determining the efficacy of an agent in treating a tumor in a subject comprising the steps of: (a) inducing tumor formation in a control subject and a test subject, wherein the Col8a1 gene has been knocked out or expression of the Col8a1 gene has been knocked down in both the control and test subjects, (b) administering the agent to the test subject, and (c) measuring the number of tumors formed and/or the size of one or more tumors in the control and test subjects, wherein the agent treats the tumor if the number of tumors in the test subject is fewer than the number of tumors in the control subject and/or the average size of the one or more tumors measured in the test subject is smaller than the average size of the one or more tumors measured in the control subject.

In some embodiments, the agent is administered to the test subject after the formation of one or more tumors in the test subject. In some embodiments, the agent is administered to the test subject before the formation of one or more tumors in the test subject. In some embodiments, the agent is administered to the test subject during the induction of tumor formation.

In some embodiments, tumor formation is induced by xenograft. Tumor formation may be induced by subcutaneous injection of cells from a tumor cell line. In some embodiments, tumor number and size are evaluated between 2 and 21 days after injection of the cells from a tumor cell line. In some embodiments, tumor number and size are evaluated 10 days after injection of the cells from a tumor cell line.

In some embodiments of any of the above aspects, the control subject and the test subject are mammals. In some embodiments of any of the above aspects, the control subject and the test subject are rodents, such as mice. In some embodiments of any of the above aspects, the control subject and the test subject are non-human primates.

In some embodiments of any of the above aspects, the agent is administered to the test subject systemically or locally. In some embodiments of any of the above aspects, the agent is systemically administered to the test subject. In some embodiments of any of the above aspects, the agent is locally administered to the test subject. In some embodiments of any of the above aspects, the agent is administered to the test subject intravitreally, intravenously, intraperitoneally, orally, subcutaneously or intramuscularly. In some embodiments of any of the above aspects, the agent is administered to the test subject intravitreally, such as by intravitreal injection or through an intravitreal device.

In some embodiments of any of the above aspects, the subject is resistant or refractory to treatment with a VEGF inhibitor, such as an anti-VEGF antibody.

In some embodiments of any of the above aspects, the method further comprises administering to the control subject and the test subject a VEGF inhibitor, such as an anti-VEGF antibody. In some embodiments, the VEGF inhibitor is administered for at least three times, for example, three times, four times, or five times. In some embodiments, the VEGF inhibitor is administered prior to administration of the agent. In some embodiments, the VEGF inhibitor is administered simultaneously as administration of the agent. In some embodiments, the VEGF inhibitor is administered daily. In some embodiments, the VEGF inhibitor is administered once every two days. In some embodiments, the VEGF inhibitor is administered once every two to three days.

In some embodiments of any of the above aspects, the test subject and control subject have a wildtype Collagen Type VIII Alpha 2 Chain (Col8a2) gene.

In a fifth aspect, the present disclosure provides a method for determining the efficacy of an agent in modulating angiogenesis or promoting wound healing in injured cells comprising the steps of: (a) injuring a control culture of cells and a test culture of cells, wherein the Collagen Type VIII Alpha 1 Chain (Col8a1) gene in the cells has been knocked out or expression of the Col8a1 gene has been knocked down in both the control and test cell cultures, (b) contacting the cells of the test culture of cells with the agent, and (c) subsequently evaluating migration of cells across the injury and/or cell proliferation in the control culture of cells and the test culture of cells, wherein the agent is effective in modulating angiogenesis if there is a difference in migration of cells across the injury and/or cell proliferation between the test culture of cells and the control culture of cells, or wherein the agent is effective in promoting wound healing if the migration of cells across the injury and/or cell proliferation is faster in the test culture of cells than in the control culture of cells.

In some embodiments, the method is for determining the efficacy of an agent in promoting angiogenesis in injured cells. In some embodiments, the control and test cells are endothelial cells, fibroblasts or retinal pigment epithelial (RPE) cells. In some embodiments, the control and test cells are vascular endothelial cells (vECs). In some embodiments, the cells are human cells. In some embodiments, the cells are differentiated from pluripotent stem cells (PSCs), such as embryonic stem cells (ESCs) or induced pluripotent stem cells (iPSCs).

In some embodiments, the injuring is a scratch. In some embodiments, the contacting step comprises adding the agent to the culture medium of the test culture of cells after the test culture of cells is injured. In some embodiments, the contacting step comprises adding the agent to the culture medium of the test culture of cells before the test culture of cells is injured.

In some embodiments of any of the above aspects, the Col8a1 gene is knocked out using gene editing technologies. In some embodiments, the gene editing technology is CRISPR/Cas9.

In some embodiments of any of the above aspects, the agent is selected from the group consisting of a small molecule, an antibody, a polypeptide, a polynucleotide, and a gene therapy. In some embodiments of any of the above aspects, the agent is not a VEGF inhibitor, such as an anti-VEGF antibody.

In some embodiments, the test culture of cells and control culture of cells have a wildtype Collagen Type VIII Alpha 2 Chain (Col8a2) gene.

In some embodiments, the cells are resistant or refractory to treatment with a VEGF inhibitor, such as an anti-VEGF antibody. In some embodiments, the method further comprises contacting the cells of the test culture and the control culture with a VEGF inhibitor, such as an anti-VEGF antibody.

Also provided are engineered non-human animals or cells in which the Collagen Type VIII Alpha 1 Chain (Col8a1) gene has been knocked out or expression of the Col8a1 gene that has been knocked down. In some embodiments, the engineered non-human animal or cell has wildtype Col8a2 gene. In some embodiments, the engineered non-human animal or cell has a mutant Col8a2, e.g., the Col8a2 gene has been knocked out or expression of the Col8a2 gene has been knocked down.

### BRIEF DESCRIPTION OF THE DRAWINGS

FIG. 1A shows the protein sequence alignment of Col8a1 wildtype (Col8a1 wt/wt) mice and Col8a1 knockout (Col8a1 ko/ko) mice. FIG. 1B shows the mean weight comparison of Col8a1 wt/wt and Col8a1 ko/ko littermates. Error bars=standard deviation. Unpaired t test p-values: ns=not statistically significant.

FIG. 2A shows an overview of the laser-induced choroidal neovascularization (CNV) mouse model. FIG. 2B shows Col8a1 (cyan) Col8a2 (magenta) expression using in situ hybridization in a resulting lesion from laser-induced CNV in a wildtype mouse.

FIGS. 3A-3C demonstrate that Col8a1 knockout (Col8a1 ko/ko) exacerbated lesion size at 7 days post laser-induced choroidal neovascularization (CNV). FIG. 3A shows the mean CNV lesion surface size in Col8a1 wild type (Col8a1 wt/wt), Col8a1 heterozygous (Col8a1 wt/ko), and Col8a1 knockout (Col8a1 ko/ko) mice. Error bars=standard deviation. ANOVA multiple comparisons p-values: *=0.0125, ****<0.0001. FIG. 3B shows the CNV lesion grading in Col8a1 wt/wt and Col8a1 ko/ko mice. Grade 0: no lesion, grade 1: lesion<0.9 μm², grade 2: lesion<1.7 μm², grade 3: lesion<2.5 μm², grade 4: lesion<3.3 μm². FIG. 3C shows the cross sections of representative CNV lesions in Col8a1 wt/wt and Col8a1 ko/ko mice eyes using hematoxylin and eosin (H&E) staining (scale bar=100 μm).

FIGS. 4A and 4B demonstrate that laser-induced choroidal neovascularization (CNV) lesion size exacerbation is caused by loss of Col8a1 and not Col8a2 at 7 days post-induction. FIG. 4A shows the CNV lesion surface size in wild type (Col8a1 wt/wt; Col8a2 wt/wt) mice, Col8a1 knockout (Col8a1 ko/ko; Col8a2 wt/wt) mice, Col8a2 knockout (Col8a1 wt/wt; Col8a2 ko/ko) mice, and Col8a1; Col8a2 double knockout (Col8a1 ko/ko; Col8a2 ko/ko) mice. Multiple comparisons test p-values: ns=not statistically significant, ****<0.0001. FIG. 4B shows the flatmounts of representative CNV lesions in wild type (Col8a1 wt/wt; Col8a2 wt/wt) mice, Col8a1 knockout (Col8a1 ko/ko; Col8a2 wt/wt) mice, Col8a2 knockout (Col8a1 wt/wt; Col8a2 ko/ko) mice, and Col8a1; Col8a2 double knockout (Col8a1 ko/ko; Col8a2 ko/ko) mice visualized using immunofluorescent staining (scale bar=100 μm). Nuclei are visualized using DAPI (blue) and blood vessels are visualized using FITC-lectin (green).

FIGS. 5A-5C demonstrate that laser-induced choroidal neovascularization (CNV) lesions in Col8a1 knockout mice show incomplete response to anti-VEGF treatment at 7 days post-induction. FIG. 5A shows the CNV lesion surface size in Col8a1 wild type (Col8a1 wt/wt) and Col8a1 knockout (Col8a1 ko/ko) mice after treatment with a control antibody (anti-gp120) or with anti-VEGF. Multiple comparisons test p-values: ns=not statistically significant, ****<0.0001. FIG. 5B shows the CNV lesion grading in Col8a1 wt/wt and Col8a1 ko/ko mice after treatment with a control antibody (anti-gp120) or with anti-VEGF. Grade 0: no lesion, grade 1: lesion<0.9 μm², grade 2: lesion<1.7 μm², grade 3: lesion<2.5 μm², grade 4: lesion<3.3 μm². FIG. 5C shows the flatmounts of representative CNV lesions in wild type (Col8a1 wt/wt) and Col8a1 knockout (Col8a1 ko/ko) mice after treatment with a control antibody (anti-gp120) or with anti-VEGF visualized using immunofluorescent staining (scale bar=100 μm). Blood vessels are visualized using FITC-lectin (green).

FIGS. 6A and 6B demonstrate that sodium iodate (NaIO₃) lesions are exacerbated by the loss of Col8a1. FIG. 6A provides optical coherence tomography (OCT) scans (upper panels) and histology (hematoxylin and eosin staining, lower panels) showing representative lesions in Col8a1 wildtype (wt/wt) and Col8a1 knockout (Col8a1ko/ko) female mice at 21 days post NaIO₃ injection (scale bar=100 μm). FIG. 6B shows the quantification of the number of retinal invaginations and grading of the severity of the lesions in Col8a1 wildtype (Col8a1 wt/wt) and Col8a1 knockout (Col8a1ko/ko) mice at 21 days post NaIO₃ injection (p-values: **=0.002, ****<0.0001).

FIG. 7A shows the generation of Col8a1 knockout (Col8a1 ko/ko) human embryonic stem cell (hESC)-derived vascular endothelial cells (vECs). Col8a1 was knocked out of hESCs via CRISPR/Cas9 and the resulting wildtype (wt/wt) and knockout (ko/ko) clones were differentiated to vECs. FIG. 7B shows the expression embryonic stem cell markers (OCT4, SOX2, NANOG and UTF1) and endothelial markers (VEGFR2, CD31, von Willebrand Factor (vWF) and VE-Cadherin) in hESC and hESC-derived vECs at different time points during the 2-week differentiation.

FIG. 8 shows the migration and proliferation of Col8a1 wildtype (Col8a1 wt/wt) and Col8a1 knockout (Col8a1 ko/ko) vECs in scratch assay.

FIG. 9 shows the migration and proliferation of Col8a1 wildtype (wt/wt) and knockout (ko/ko) vECs in scratch assay in response to anti-VEGF antibody treatment.

### DETAILED DESCRIPTION

### General

Practice of the methods disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature.

The term “herein” means the entire application.

It should be understood that any of the embodiments described herein, including those described under different aspects of the disclosure and different parts of the specification (including embodiments described only in the Examples) can be combined with one or more other embodiments disclosed herein, unless explicitly disclaimed or improper. Combination of embodiments are not limited to those specific combinations claimed via the multiple dependent claims.

Any publications, patents and published patent applications referred to in this application are specifically incorporated by reference herein. In case of conflict, the present specification, including its specific definitions, will control.

Throughout this specification, the word “comprise,” or variations such as “comprises” or “comprising,” which is synonymous with “including,” “containing,” or “characterized by,” is inclusive or open-ended and does not exclude additional, unrecited elements or method steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Similarly, where methods or processes are described as having, including, or comprising specific process steps, the processes also may consist essentially of, or consist of, the recited processing steps. Further, it should be understood that, unless otherwise indicated or the context clearly indicates otherwise, the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

As used herein, the term “about” modifying the quantity of an ingredient, parameter, calculation, or measurement in the compositions employed in the methods of the disclosure refers to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be typically within 10%, more typically still within 5%, of a given value or range. The term “about” also encompasses amounts that differ due to different equilibrium conditions for a composition resulting from a particular initial mixture. Whether or not modified by the term “about,” the paragraphs include equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range.

The term “or” as used herein should be understood to mean “and/or,” unless the context clearly indicates otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

Exemplary methods and materials are described herein, although methods and materials similar or equivalent to those described herein can also be used in the practice or testing of the present application. The materials, methods, and examples are illustrative only and not intended to be limiting.

### Definitions

The following terms, unless otherwise indicated, shall be understood to have the following meanings:

As used herein, the term “agent” is used to denote a chemical compound (such as an organic or inorganic compound), a mixture of chemical compounds, a biological macromolecule (such as a polynucleotide, an antibody, a protein or portion thereof, e.g., a peptide, a lipid, or a carbohydrate) or an extract made from biological materials such as bacteria, plants, fungi, or animal (particularly mammalian) cells or tissues. Agents include, for example, compounds which are known with respect to structure and/or function, and those which are not known with respect to structure or function. The activity of such agents may render it suitable as a “therapeutic agent” which is a biologically, physiologically, or pharmacologically active substance (or substances) that acts locally or systemically in a subject. Agents can comprise, for example, drugs, metabolites, intermediates, cofactors, transition state analogs, ions, metals, toxins and natural and synthetic polymers (e.g., proteins, peptides, polynucleotides, polysaccharides, glycoproteins, hormones, receptors and cell surfaces such as cell walls and cell membranes). Agents may also comprise alcohols, alkyl halides, amines, amides, esters, aldehydes, ethers and other classes of organic agents.

“Administering” or “administration of” a substance, a compound or an agent to a subject refers to the contact of that substance, compound or agent to the subject or a cell, tissue, organ or bodily fluid of the subject. For example, a compound or an agent can be administered intravitreally. Administering can also be performed, for example, once, a plurality of times, and/or over one or more extended periods.

As used herein, the terms “treat”, “treating” and “treatment” refer to the administration of a therapeutic agent to a subject or patient having one or more disease symptoms, or being suspected of having a disease, for which the agent has therapeutic activity. “Treat”, “treating” and “treatment” include, e.g., a method of alleviating or reducing the severity of a condition or abolishing a condition and includes alleviating or reducing the severity of one or more symptoms of the condition. The alleviation or reduction of a disease symptom can be assessed by any clinical measurement typically used by physicians or other skilled artisans to assess the severity or progression of that symptom. The terms further refer to a postponement of development of one or more disease symptoms and/or a reduction in the severity of one or more disease symptoms. The terms further include ameliorating existing uncontrolled or unwanted disease symptoms, preventing additional disease symptoms, and ameliorating or preventing the underlying causes of such disease symptoms. Thus, the terms denote that a beneficial result has been conferred on the subject.

As used herein the term “subject” refers to animals, including mammals. In some embodiments, the subject is a rodent (e.g., a mouse or a rat). Additional non-limiting examples of subjects include non-human primates and domesticated animals, including dogs, cats, sheep, cattle, horses, goats, pigs, mice, rats, rabbits, hamsters, and guinea pigs.

As used herein the term “tissue” refers to an organ or set of specialized cells that function together as a unit. Non-limiting examples of tissues include a retina, skin, and kidney.

As used herein, the term “polynucleotide” refers to a polymer of nucleic acid residues. In some embodiments, the polynucleotide comprises deoxyribonucleic acid (DNA) residues. In some embodiments, the polynucleotide comprises ribonucleic acid (RNA) residues. In some embodiments, the polynucleotide comprises DNA and RNA residues. The term should also be understood to include, as equivalents, analogs of either RNA or DNA made from nucleotide analogs, and, as applicable to the embodiment being described, single-stranded (such as sense or antisense) and double-stranded polynucleotide. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, small interfering RNA (siRNA), micro-RNA, guide RNA (gRNA) cDNA, recombinant polynucleotides, branched polynucleotides, plasmids, vectors, isolated DNA of any sequence, isolated RNA of any sequence, probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. The term “recombinant” polynucleotide means a polynucleotide of genomic, cDNA, semi-synthetic, or synthetic origin which either does not occur in nature or is linked to another polynucleotide in a non-natural arrangement. The polynucleotide may be operatively linked to an “expression control sequence,” which refers to a nucleotide sequence that regulates the expression of a gene.

The terms “peptides”, “proteins” and “polypeptides” are used interchangeably herein.

As used herein, “angiogenesis” refers to the growth of new blood vessels form from the existing vasculature.

As used herein, “choroidal neovascularization” or “CNV” refers to the abnormal or pathologic growth of new blood vessels originating from the choroid layer of the eye into the retinal pigment epithelium, the space beneath the retina, or the retina. Unlike normal blood vessels, the new blood vessels in CNV are leaky, allowing fluid from the blood, and sometimes red blood cells, to enter the retina, resulting in vision distortion and cell death throughout the retina, particularly to the photoreceptors.

As used herein, “gene editing” or “genome editing” refers to alteration of the genetic material of a cell or living organism by inserting, replacing, or deleting a DNA sequence with the aim of changing the activity of a gene. Examples of gene editing technologies include, without limitations, zinc finger nucleases (ZFNs), transcription activator-like effector nucleases (TALENs), meganucleases (such as MegaTALs), and clustered regularly interspaced short palindromic repeats (CRISPR)/Cas systems. Non-limiting examples of Cas proteins include Cas9, Cas12a and Cas13. In some embodiments, the gene editing technology is CRISPR/Cas9.

### Engineered Animals and Cells

Collagen type VIII Alpha 1 Chain (Col8a1) is a member of the nonfibrillar short-chain collagen family and is a component of many extracellular matrices, modulating diverse cellular responses such as proliferation, adhesion, migration, chemotaxis, and metalloproteinase synthesis (Hopfer U., et al. Diabetes. 58(7):1672-81, 2009 and Hou G., et al. J Clin Invest. 107(6):727-35, 2001). The data presented herein unexpectedly demonstrate that knockdown/knockout of the Col8a1 gene in mice and cells makes them less responsive to VEGF inhibitors. Accordingly, such Col8a1 knockdown/knockout cells and animals may provide more a more clinically accurate model of neovascularization and provide an improved model for evaluating the efficacy of therapies, including non-VEGF inhibitor therapies, for treating angiogenesis-associated diseases and disorders.

In some embodiments, the engineered animal or cell has its Col8a1 gene knocked out. In some embodiments, the engineered animal or cell is homozygous with a Col8a1 loss-of-function mutation (i.e., Col8a1−/−). In some embodiments, expression of Col8a1 in the engineered animal or cell has been knocked down. In some embodiments, the engineered animal or cell is heterozygous with a Col8a1 loss-of-function mutation (i.e., Col8a1+/−). In some embodiments, the engineered animal or cell has wildtype Collagen type VIII Alpha 2 Chain (i.e., Col8a2+/+). In some embodiments, the engineered animal or cell has its Col8a2 gene knocked out. In some embodiments, the engineered animal or cell is homozygous with a Col8a2 loss-of-function mutation (i.e., Col8a2−/−). In some embodiments, the engineered animal or cell is heterozygous with a Col8a2 loss-of-function mutation (i.e., Col8a2+/−).

In some embodiments, the engineered animal or cell is Col8a1−/− Col8a2+/+. In some embodiments, the engineered animal or cell is Col8a1+/+ Col8a2+/+, wherein expression of the Col8a1 gene has been knocked down.

In some embodiments, the Col8a1 gene is knocked out using gene editing technologies. Any gene editing technology known in the art for making a knockout can be used. Non-limiting gene editing technologies for knocking out a gene include homologous recombination, site-specific recombination (e.g. Cre-Lox and Flp-Frt) systems, and CRISPR. In some embodiments, the gene editing technology is a CRISPR-based system, such as CRISPR/Cas9. Any other suitable CRISPR system (e.g., Cas12a and Cas13) may also be used. In some embodiments, the CRISPR system uses a pair of single-guide RNAs (sgRNAs). In some embodiments, the sgRNAs targets sequences in exon 4 of the Col8a1 gene. In some embodiments, the sgRNA comprises the nucleic acid sequence of SEQ ID NO: 1. In some embodiments, the sgRNA comprises the nucleic acid sequence of SEQ ID NO: 2. In some embodiments, one of the sgRNA comprises the nucleic acid sequence of SEQ ID NO: 1 and the other sgRNA comprises the nucleic acid sequence of SEQ ID NO: 2. In some embodiments, the sgRNA is electroporated into a zygote which grows into the test subject or an ancestor of the test subject. See, e.g., Modzelewski et al. (2018) Nat Protoc. 13: 1253-1274. In some embodiments, the sgRNA is transcribed in vitro before electroporation.

In some embodiments, the engineered Col8a1 gene has a deletion in exon 4. In some embodiments, the engineered Col8a1 gene lacks a nucleic acid sequence encoding amino acid residues G110 to R130 of SEQ ID NO: 6. In some embodiments, the engineered animal of cell has a 2150 bp knockout region corresponding to GRCm38/mm10 chr16: 57,631,573-57,633,722.

In some embodiments, the engineered animal or cell comprises in its genome a nucleic acid sequence encoding the amino acid sequence of SEQ ID NO: 7. In some embodiments, the engineered animal has a germline genomic sequence comprising a nucleic acid sequence encoding the amino acid sequence of SEQ ID NO: 7.

In some embodiments, expression of the Col8a1 gene is knocked down. Any technology for knocking down the expression of a gene known in the art may be used. Non-limiting examples of technology for knocking down the expression of a gene include RNAi (e.g., siRNA and miRNA) and CRISPR. In some embodiments, the expression of the Col8a1 gene is knocked down at least 10%. In some embodiments, the expression of the Col8a1 gene is knocked down at least 20%. In some embodiments, the expression of the Col8a1 gene is knocked down at least 30%. In some embodiments, the expression of the Col8a1 gene is knocked down at least 40%. In some embodiments, the expression of the Col8a1 gene is knocked down at least 50%. In some embodiments, the expression of the Col8a1 gene is knocked down at least 60%. In some embodiments, the expression of the Col8a1 gene is knocked down at least 70%. In some embodiments, the expression of the Col8a1 gene is knocked down at least 80%. In some embodiments, the expression of the Col8a1 gene is knocked down at least 90%. In some embodiments, the expression of the Col8a1 gene is knocked down at least 95%. In some embodiments, the expression of the Col8a1 gene is knocked down at least 96%. In some embodiments, the expression of the Col8a1 gene is knocked down at least 97%. In some embodiments, the expression of the Col8a1 gene is knocked down at least 98%. In some embodiments, the expression of the Col8a1 gene is knocked down at least 99%.

In some embodiments, the engineered animal is a mammal. In some embodiments, the engineered animal is a rodent. In some embodiments, the engineered animal is a mouse. In some embodiments, the engineered animal is a rat. In some embodiments, the engineered animal is a non-human primate. In some embodiments, the engineered animal a domesticated animal, such as a dog, a cat, a sheep, a cow, a horse, a goat, a pig, a mouse, a rat, a rabbit, a hamster, and a guinea pig. In some embodiments, the engineered animal is a dog. In some embodiments, the engineered animal is a cat. In some embodiments, the engineered animal is a sheep. In some embodiments, the engineered animal is a cow. In some embodiments, the engineered animal is a horse. In some embodiments, the engineered animal is a goat. In some embodiments, the engineered animal is a pig. In some embodiments, the engineered animal is a rabbit. In some embodiments, the engineered animal is a hamster. In some embodiments, the engineered animal is a guinea pig.

In some embodiments, the engineered cell is a mammalian cell. In some embodiments, the engineered cell is a human cell. In some embodiments, the engineered cell is a rodent cell. In some embodiments, the engineered cell is a mouse cell. In some embodiments, the engineered cell is a rat cell. In some embodiments, the engineered cell is a non-human primate cell. In some embodiments, the engineered cell a domesticated animal cell. In some embodiments, the engineered cell is a dog cell. In some embodiments, the engineered cell is a cat cell. In some embodiments, the engineered cell is a sheep cell. In some embodiments, the engineered cell is a cow cell. In some embodiments, the engineered cell is a horse cell. In some embodiments, the engineered cell is a goat cell. In some embodiments, the engineered cell is a pig cell. In some embodiments, the engineered cell is a rabbit cell. In some embodiments, the engineered cell is a hamster cell. In some embodiments, the engineered cell is a guinea pig cell.

### In Vivo Methods

The present disclosure provides methods of modeling angiogenesis-associated diseases or conditions in a tissue of a subject, e.g., a subject that is resistant or refractory to treatment with a VEGF inhibitor, such as an anti-VEGF antibody. The methods utilize an engineered animal described herein in which the Col8a1 gene has been knocked out or in which expression of Col8a1 gene has been knockdown and comprise the step of inducing neovascularization in the tissue of the animal.

In a first aspect, the present disclosure provides a method for determining the efficacy of an agent in modulating angiogenesis in a tissue. In some embodiments, the method comprises the steps of: (a) inducing neovascularization in the tissue of a control subject and a test subject, wherein the Collagen Type VIII Alpha 1 Chain (Col8a1) gene has been knocked out or expression of the Col8a1 gene has been knocked down in both the control and test subjects, (b) administering the agent to the test subject, and (c) measuring the size of one or more lesions resulting from the neovascularization in the control and test subjects, wherein the agent modulates angiogenesis in the tissue if the size of the one or more lesions in the test subject is smaller than the size of the one or more lesions in the control subject.

In some embodiments, the agent modulates angiogenesis if the average lesion size in the test subject is at least 10% smaller than the average lesion size in the control subject. In some embodiments, the agent modulates angiogenesis if the average lesion size in the test subject is at least 20% smaller than the average lesion size in the control subject. In some embodiments, the agent modulates angiogenesis if the average lesion size in the test subject is at least 30% smaller than the average lesion size in the control subject. In some embodiments, the agent modulates angiogenesis if the average lesion size in the test subject is at least 40% smaller than the average lesion size in the control subject. In some embodiments, the agent modulates angiogenesis if the average lesion size in the test subject is at least 50% smaller than the average lesion size in the control subject. In some embodiments, the agent modulates angiogenesis if the average lesion size in the test subject is at least 60% smaller than the average lesion size in the control subject. In some embodiments, the agent modulates angiogenesis if the average lesion size in the test subject is at least 70% smaller than the average lesion size in the control subject. In some embodiments, the agent modulates angiogenesis if the average lesion size in the test subject is at least 80% smaller than the average lesion size in the control subject. In some embodiments, the agent modulates angiogenesis if the average lesion size in the test subject is at least 90% smaller than the average lesion size in the control subject. In some embodiments, the agent modulates angiogenesis if the average lesion size in the test subject is at least 95% smaller than the average lesion size in the control subject. In some embodiments, the agent modulates angiogenesis if the lesions in the test subject are completely resolved compared to the control subject.

In some embodiments, the tissue is retina. In some embodiments, the tissue is a tumor.

Any method of inducing neovascularization known in the art may be used. In some embodiments, the neovascularization is induced by application of a laser to the basement membrane of an eye of the control subject and the test subject. In some embodiments, the neovascularization is induced by transplantation of a cell matrix plug, e.g., a Matrigel plug, into the control subject and the test subject. In some embodiments, the neovascularization is induced by xenograft into the control subject and the test subject.

In some embodiments, the agent is administered to the test subject after neovascularization is induced. In some embodiments, the agent is administered to the test subject before neovascularization is induced. In some embodiments, the agent is administered to the test subject during the induction of the neovascularization.

In some embodiments, the method further comprises administering to the control subject and the test subject a VEGF inhibitor, such as an anti-VEGF antibody. In some embodiments, the VEGF inhibitor is administered for at least three times, for example, three times, four times, or five times. In some embodiments, the VEGF inhibitor is administered three times. In some embodiments, the VEGF inhibitor is administered four times. In some embodiments, the VEGF inhibitor is administered five times. In some embodiments, the VEGF inhibitor is administered prior to administration of the agent. In some embodiments, the VEGF inhibitor is administered simultaneously as administration of the agent. In some embodiments, the VEGF inhibitor is administered daily. In some embodiments, the VEGF inhibitor is administered on the same day, one day, two days, three days, four days, and five days after the induction of the neovascularization. In some embodiments, the VEGF inhibitor is administered once every two to three days. In some embodiments, the VEGF inhibitor is administered once every two days. In some embodiments, the VEGF inhibitor is administered once every three days. In some embodiments, the VEGF inhibitor is administered on the same day, two days and five days after the induction of the neovascularization. In some embodiments, the VEGF inhibitor is administered on the same day, three days and five days after the induction of the neovascularization. In some embodiments, the VEGF inhibitor is administered systemically, such as intraperitoneally. In some embodiments, the VEGF inhibitor is administered locally, such as intravitreally.

The present disclosure also provides methods of modeling wound healing or fibrosis in an injured tissue of a subject, e.g., a subject that is resistant or refractory to treatment with a VEGF inhibitor, such as an anti-VEGF antibody. The methods utilize an engineered animal described herein in which the Col8a1 gene has been knocked out or in which expression of Col8a1 gene has been knockdown and comprise the step of inducing one or more lesions by injuring the tissue of the animal.

In a second aspect, the present disclosure provides a method for determining the efficacy of an agent in promoting wound healing or inhibiting fibrosis of an injured tissue in a subject. In some embodiments the method comprises the steps of: (a) inducing one or more lesions by injuring the tissue of a control subject and a test subject, wherein the Collagen Type VIII Alpha 1 Chain (Col8a1) gene has been knocked out or expression of the Col8a1 gene has been knocked down in both the control and test subjects, (b) administering the agent to the test subject, and (c) measuring the size of the one or more lesions and/or one or more fibrotic scars formed at the one or more lesions in the control and test subjects, wherein the agent promotes wound healing in the injured tissue if the size of the one or more lesions in the test subject is smaller than the size of the one or more lesions in the control subject, and/or if the size of the one or more fibrotic scars in the test subject is smaller than the size of the one or more fibrotic scars in the test subject; and wherein the agent inhibits fibrosis of the injured tissue if the size of the one or more fibrotic scars in the test subject is smaller than the size of the one or more fibrotic scars in the test subject.

In some embodiments, the agent promotes wound healing if the average lesion size in the test subject is at least 10% smaller than the average lesion size in the control subject. In some embodiments, the agent promotes wound healing if the average lesion size in the test subject is at least 20% smaller than the average lesion size in the control subject. In some embodiments, the agent promotes wound healing if the average lesion size in the test subject is at least 30% smaller than the average lesion size in the control subject. In some embodiments, the agent promotes wound healing if the average lesion size in the test subject is at least 40% smaller than the average lesion size in the control subject. In some embodiments, the agent promotes wound healing if the average lesion size in the test subject is at least 50% smaller than the average lesion size in the control subject. In some embodiments, the agent promotes wound healing if the average lesion size in the test subject is at least 60% smaller than the average lesion size in the control subject. In some embodiments, the agent promotes wound if the average lesion size in the test subject is at least 70% smaller than the average lesion size in the control subject. In some embodiments, the agent promotes wound healing if the average lesion size in the test subject is at least 80% smaller than the average lesion size in the control subject. In some embodiments, the agent promotes wound healing if the average lesion size in the test subject is at least 90% smaller than the average lesion size in the control subject. In some embodiments, the agent promotes wound healing if the average lesion size in the test subject is at least 95% smaller than the average lesion size in the control subject. In some embodiments, the agent promotes wound healing if the lesions in the test subject are completely resolved compared to the control subject.

In some embodiments, the agent promotes wound healing or inhibits fibrosis if the average size of the fibrotic scar in the test subject is at least 10% smaller than the average size of the fibrotic scar in the control subject. In some embodiments, the agent promotes wound healing or inhibits fibrosis if the average size of the fibrotic scar in the test subject is at least 20% smaller than the average size of the fibrotic scar in the control subject. In some embodiments, the agent promotes wound healing or inhibits fibrosis if the average size of the fibrotic scar in the test subject is at least 30% smaller than the average size of the fibrotic scar in the control subject. In some embodiments, the agent promotes wound healing or inhibits fibrosis if the average size of the fibrotic scar in the test subject is at least 40% smaller than the average size of the fibrotic scar in the control subject. In some embodiments, the agent promotes wound healing or inhibits fibrosis if the average size of the fibrotic scar in the test subject is at least 50% smaller than the average size of the fibrotic scar in the control subject. In some embodiments, the agent promotes wound healing or inhibits fibrosis if the average size of the fibrotic scar in the test subject is at least 60% smaller than the average size of the fibrotic scar in the control subject. In some embodiments, the agent promotes wound healing or inhibits fibrosis if the average size of the fibrotic scar in the test subject is at least 70% smaller than the average size of the fibrotic scar in the control subject. In some embodiments, the agent promotes wound healing or inhibits fibrosis if the average size of the fibrotic scar in the test subject is at least 80% smaller than the average size of the fibrotic scar in the control subject. In some embodiments, the agent promotes wound healing or inhibits fibrosis if the average size of the fibrotic scar in the test subject is at least 90% smaller than the average size of the fibrotic scar in the control subject. In some embodiments, the agent promotes wound healing or inhibits fibrosis if the average size of the fibrotic scar in the test subject is at least 95% smaller than the average size of the fibrotic scar in the control subject. In some embodiments, the agent promotes wound healing or inhibits fibrosis if the test subject forms no fibrotic scars compared to the control subject.

In some embodiments, the injured tissue is skin or kidney. In some embodiments, the injured tissue is skin. In some embodiments, the injured tissue is kidney. In some embodiments, the injured tissue is retina.

Any method of inducing tissue injury known in the art may be used. In some embodiments, the tissue injury is caused by surgical incision or skin biopsy punch. In some embodiments, the tissue injury is caused by surgical incision. In some embodiments, the tissue injury is induced by skin biopsy punch.

In some embodiments, the agent is administered to the test subject after the one or more lesions are induced. In some embodiments, the agent is administered to the test subject before the one or more lesions are induced. In some embodiments, the agent is administered to the test subject during the induction of the one or more lesions.

In some embodiments, the method further comprises administering to the control subject and the test subject a VEGF inhibitor, such as an anti-VEGF antibody. In some embodiments, the VEGF inhibitor is administered for at least three times, for example, three times, four times, or five times. In some embodiments, the VEGF inhibitor is administered three times. In some embodiments, the VEGF inhibitor is administered four times. In some embodiments, the VEGF inhibitor is administered five times. In some embodiments, the VEGF inhibitor is administered prior to administration of the agent. In some embodiments, the VEGF inhibitor is administered simultaneously as administration of the agent. In some embodiments, the VEGF inhibitor is administered once every two to three days. In some embodiments, the VEGF inhibitor is administered once every two days. In some embodiments, the VEGF inhibitor is administered once every three days. In some embodiments, the VEGF inhibitor is administered systemically, such as intraperitoneally. In some embodiments, the VEGF inhibitor is administered locally.

The present disclosure further provides methods of modeling age-related macular degeneration (AMD) in a subject, e.g., a subject that is resistant or refractory to treatment with a VEGF inhibitor, such as an anti-VEGF antibody. The methods utilize an engineered animal described herein in which the Col8a1 gene has been knocked out or in which expression of Col8a1 gene has been knockdown and comprise the step of inducing one or more lesions in the retina of the animal.

In a third aspect, the present disclosure provides a method for determining the efficacy of an agent in treating or preventing age-related macular degeneration (AMD) in a subject. In some embodiments, the method comprises the steps of: (a) inducing one or more lesions in the retina of a control subject and a test subject, wherein the Collagen Type VIII Alpha 1 Chain (Col8a1) gene has been knocked out or expression of the Col8a1 gene has been knocked down in both the control and test subjects, (b) administering the agent to the test subject, and (c) measuring the size of one or more lesions in the control and test subjects, wherein the agent treats or prevents AMD if the size of the one or more lesions in the test subject is smaller than the size of the one or more lesions in the control subject.

In some embodiments, the agent treats or prevents AMD if the average lesion size in the test subject is at least 10% smaller than the average lesion size in the control subject. In some embodiments, the agent treats or prevents AMD if the average lesion size in the test subject is at least 20% smaller than the average lesion size in the control subject. In some embodiments, the agent treats or prevents AMD if the average lesion size in the test subject is at least 30% smaller than the average lesion size in the control subject. In some embodiments, the agent treats or prevents AMD if the average lesion size in the test subject is at least 40% smaller than the average lesion size in the control subject. In some embodiments, the agent treats or prevents AMD if the average lesion size in the test subject is at least 50% smaller than the average lesion size in the control subject. In some embodiments, the agent treats or prevents AMD if the average lesion size in the test subject is at least 60% smaller than the average lesion size in the control subject. In some embodiments, the agent treats or prevents AMD if the average lesion size in the test subject is at least 70% smaller than the average lesion size in the control subject. In some embodiments, the agent treats or prevents AMD if the average lesion size in the test subject is at least 80% smaller than the average lesion size in the control subject. In some embodiments, the agent treats or prevents AMD if the average lesion size in the test subject is at least 90% smaller than the average lesion size in the control subject. In some embodiments, the agent treats or prevents AMD if the average lesion size in the test subject is at least 95% smaller than the average lesion size in the control subject. In some embodiments, the agent treats or prevents AMD if the lesions in the test subject are completely resolved compared to the control subject.

In some embodiments, the AMD is neovascular AMD. In some embodiments, the AMD is geographic atrophy (GA).

Any method of inducing lesion formation in the eye known in the art may be used. In some embodiments, the one or more lesions is induced by application of a laser to the basement membrane of an eye of the control subject and the test subject. In some embodiments, the one or more lesions is induced by injection of sodium iodate into the control subject and the test subject. In some embodiments, the sodium iodate is administered locally. In some embodiments, the sodium iodate is administered intravitreally or retro-orbitally. In some embodiments, the sodium iodate is administered intravitreally. In some embodiments, the sodium iodate is administered retro-orbitally. In some embodiments, the sodium iodate is administered systemically. In some embodiments, the sodium iodate is administered intravenously.

In some embodiments, the agent is administered to the test subject after the one or more lesions are induced. In some embodiments, the agent is administered to the test subject before the one or more lesions are induced. In some embodiments, the agent is administered to the test subject during the induction of the one or more lesions. In some embodiments, the agent is administered to the test subject intravitreally. In some embodiments, the agent is administered to the test subject by intravitreal injection. In some embodiments, the agent is administered to the test subject through an intravitreal device.

In some embodiments, the lesion size is measured between 5 and 10 days after the one or more lesions in the retina is induced. In some embodiments, the lesion size is measured between 6 and 8 days after the one or more lesions in the retina is induced. The lesion size may be measured 5 days after the one or more lesions in the retina is induced. The lesion size may be measured 6 days after the one or more lesions in the retina is induced. The lesion size may be measured 7 days after the one or more lesions in the retina is induced. The lesion size may be measured 8 days after the one or more lesions in the retina is induced. The lesion size may be measured 9 days after the one or more lesions in the retina is induced. The lesion size may be measured 10 days after the one or more lesions in the retina is induced.

In some embodiments, the lesion size is identified by fluorescent, histological and/or optical coherence tomography analysis, such as by fluorescent microscopy. Non-limiting examples of methods of evaluating an AMD lesion include optical coherence tomography (OCT), polarization-sensitive spectral-domain optical coherence tomography (PS-OCT), scanning laser ophthalmoscopy (SLO), fundus autofluorescence (FAF), near-infrared autofluorescence (NI-AF) imaging, fluorescein angiography (FA), indocyanine green angiography (ICGA), and intensity-based spectral-domain OCT (SD-OCT). Fluorescein angiography is the most sensitive and widely used method to diagnose wet AMD. In some embodiments, the lesion size is identified by fluorescent analysis. In some embodiments, the lesion size is identified by histological analysis. In some embodiments, the lesion size is identified by optical coherence tomography analysis. In some embodiments, the lesion size is identified by fluorescent, histological or optical coherence tomography analysis, or any combination thereof.

In some embodiments, the method further comprises administering to the control subject and the test subject a VEGF inhibitor, such as an anti-VEGF antibody. In some embodiments, the VEGF inhibitor is administered for at least three times, for example, three times, four times, or five times. In some embodiments, the VEGF inhibitor is administered three times. In some embodiments, the VEGF inhibitor is administered four times. In some embodiments, the VEGF inhibitor is administered five times. In some embodiments, the VEGF inhibitor is administered prior to administration of the agent. In some embodiments, the VEGF inhibitor is administered simultaneously as administration of the agent. In some embodiments, the VEGF inhibitor is administered daily. In some embodiments, the VEGF inhibitor is administered on the same day, one day, two days, three days, four days, and five days after the induction of the neovascularization. In some embodiments, the VEGF inhibitor is administered once every two to three days. In some embodiments, the VEGF inhibitor is administered once every two days. In some embodiments, the VEGF inhibitor is administered once every three days. In some embodiments, the VEGF inhibitor is administered on the same day, two days and five days after the induction of the neovascularization. In some embodiments, the VEGF inhibitor is administered on the same day, three days and five days after the induction of the neovascularization. In some embodiments, the VEGF inhibitor is administered systemically, such as intraperitoneally. In some embodiments, the VEGF inhibitor is administered locally, such as intravitreally.

The present disclosure further provides methods of modeling tumor development in a subject, e.g., a subject that is resistant or refractory to treatment with a VEGF inhibitor, such as an anti-VEGF antibody. The methods utilize an engineered animal described herein in which the Col8a1 gene has been knocked out or in which expression of Col8a1 gene has been knockdown and comprise the step of inducing tumor formation the animal.

In a fourth aspect, the present disclosure provides a method for determining the efficacy of an agent in treating a tumor in a subject comprising the steps of: (a) inducing tumor formation in a control subject and a test subject, wherein the Col8a1 gene has been knocked out or expression of the Col8a1 gene has been knocked down in both the control and test subjects, (b) administering the agent to the test subject, and (c) measuring the number of tumors formed and/or the size of one or more tumors in the control and test subjects, wherein the agent treats the tumor if the number of tumors in the test subject is fewer than the number of tumors in the control subject and/or the average size of the one or more tumors measured in the test subject is smaller than the average size of the one or more tumors measured in the control subject.

In some embodiments, step (c) comprises measuring the number of tumors formed, wherein the agent treats the tumor if the number of tumors in the test subject is fewer than the number of tumors in the control subject. In some embodiments, the agent treats the tumor if the average number of tumors in the test subject is at least 10% less than the average number of tumors in the control subject. In some embodiments, the agent treats the tumor if the average number of tumors in the test subject is at least 20% less than the average number of tumors in the control subject. In some embodiments, the agent treats the tumor if the average number of tumors in the test subject is at least 30% less than the average number of tumors in the control subject. In some embodiments, the agent treats the tumor if the average number of tumors in the test subject is at least 40% less than the average number of tumors in the control subject. In some embodiments, the agent treats the tumor if the average number of tumors in the test subject is at least 50% less than the average number of tumors in the control subject. In some embodiments, the agent treats the tumor if the average number of tumors in the test subject is at least 60% less than the average number of tumors in the control subject. In some embodiments, the agent treats the tumor if the average number of tumors in the test subject is at least 70% less than the average number of tumors in the control subject. In some embodiments, the agent treats the tumor if the average number of tumors in the test subject is at least 80% less than the average number of tumors in the control subject. In some embodiments, the agent treats the tumor if the average number of tumors in the test subject is at least 90% less than the average number of tumors in the control subject. In some embodiments, the agent treats the tumor if the average number of tumors in the test subject is at least 95% less than the average number of tumors in the control subject. In some embodiments, the agent treats the tumor if the average number of tumors in the test subject is at least 99% less than the average number of tumors in the control subject.

In some embodiments, step (c) comprises measuring the size of one or more tumors in the control and test subjects, wherein the agent treats the tumor if the average size of the one or more tumors measured in the test subject is smaller than the average size of the one or more tumors measured in the control subject. In some embodiments, the agent treats the tumor if the average tumor size in the test subject is at least 10% smaller than the average tumor size in the control subject. In some embodiments, the agent treats the tumor if the average tumor size in the test subject is at least 20% smaller than the average tumor size in the control subject. In some embodiments, the agent treats the tumor if the average tumor size in the test subject is at least 30% smaller than the average tumor size in the control subject. In some embodiments, the agent treats the tumor if the average tumor size in the test subject is at least 40% smaller than the average tumor size in the control subject. In some embodiments, the agent treats the tumor if the average tumor size in the test subject is at least 50% smaller than the average tumor size in the control subject. In some embodiments, the agent treats the tumor if the average tumor size in the test subject is at least 60% smaller than the average tumor size in the control subject. In some embodiments, the agent treats the tumor if the average tumor size in the test subject is at least 70% smaller than the average tumor size in the control subject. In some embodiments, the agent treats the tumor if the average tumor size in the test subject is at least 80% smaller than the average tumor size in the control subject. In some embodiments, the agent treats the tumor if the average tumor size in the test subject is at least 90% smaller than the average tumor size in the control subject. In some embodiments, the agent treats the tumor if the average tumor size in the test subject is at least 95% smaller than the average tumor size in the control subject. In some embodiments, the agent treats the tumor if the average tumor size in the test subject is at least 99% smaller than the average tumor size in the control subject.

In some embodiments, the agent is administered to the test subject after the formation of one or more tumors in the test subject. In some embodiments, the agent is administered to the test subject before the formation of one or more tumors in the test subject. In some embodiments, the agent is administered to the test subject during the induction of tumor formation.

Any method of tumor formation known in the art may be used. In some embodiments, tumor formation is induced by xenograft. In some embodiments, tumor formation may be induced by subcutaneous injection of cells from a tumor cell line. In some embodiments, tumor number and size are evaluated between 2 and 21 days after injection of the cells from a tumor cell line. In some embodiments, tumor number and size are evaluated between 6 and 8 days after injection of the cells from a tumor cell line. In some embodiments, tumor number and size are evaluated between 7 and 10 days after injection of the cells from a tumor cell line. In some embodiments, tumor number and size are evaluated between 13 and 15 days after injection of the cells from a tumor cell line. In some embodiments, tumor number and size are evaluated between 14 and 17 days after injection of the cells from a tumor cell line. In some embodiments, tumor number and size are evaluated between 18 and 21 days after injection of the cells from a tumor cell line. In some embodiments, tumor number and size are evaluated 2 days after injection of the cells from a tumor cell line. In some embodiments, tumor number and size are evaluated 3 days after injection of the cells from a tumor cell line. In some embodiments, tumor number and size are evaluated 4 days after injection of the cells from a tumor cell line. In some embodiments, tumor number and size are evaluated 5 days after injection of the cells from a tumor cell line. In some embodiments, tumor number and size are evaluated 6 days after injection of the cells from a tumor cell line. In some embodiments, tumor number and size are evaluated 7 days after injection of the cells from a tumor cell line. In some embodiments, tumor number and size are evaluated 8 days after injection of the cells from a tumor cell line. In some embodiments, tumor number and size are evaluated 9 days after injection of the cells from a tumor cell line. In some embodiments, tumor number and size are evaluated 10 days after injection of the cells from a tumor cell line. In some embodiments, tumor number and size are evaluated 11 days after injection of the cells from a tumor cell line. In some embodiments, tumor number and size are evaluated 12 days after injection of the cells from a tumor cell line. In some embodiments, tumor number and size are evaluated 13 days after injection of the cells from a tumor cell line. In some embodiments, tumor number and size are evaluated 14 days after injection of the cells from a tumor cell line. In some embodiments, tumor number and size are evaluated 15 days after injection of the cells from a tumor cell line. In some embodiments, tumor number and size are evaluated 16 days after injection of the cells from a tumor cell line. In some embodiments, tumor number and size are evaluated 17 days after injection of the cells from a tumor cell line. In some embodiments, tumor number and size are evaluated 18 days after injection of the cells from a tumor cell line. In some embodiments, tumor number and size are evaluated 19 days after injection of the cells from a tumor cell line. In some embodiments, tumor number and size are evaluated 20 days after injection of the cells from a tumor cell line. In some embodiments, tumor number and size are evaluated 21 days after injection of the cells from a tumor cell line.

In some embodiments, the method further comprises administering to the control subject and the test subject a VEGF inhibitor, such as an anti-VEGF antibody. In some embodiments, the VEGF inhibitor is administered for at least three times, for example, three times, four times, or five times. In some embodiments, the VEGF inhibitor is administered three times. In some embodiments, the VEGF inhibitor is administered four times. In some embodiments, the VEGF inhibitor is administered five times. In some embodiments, the VEGF inhibitor is administered prior to administration of the agent. In some embodiments, the VEGF inhibitor is administered simultaneously as administration of the agent. In some embodiments, the VEGF inhibitor is administered daily. In some embodiments, the VEGF inhibitor is administered once every two to three days. In some embodiments, the VEGF inhibitor is administered once every two days. In some embodiments, the VEGF inhibitor is administered once every three days. In some embodiments, the VEGF inhibitor is administered systemically, such as intraperitoneally. In some embodiments, the VEGF inhibitor is administered locally, such as intratumorally.

In some embodiments of any of the above aspects, the control subject and the test subject are any of the engineered animals disclosed herein. In some embodiments of any of the above aspects, the control subject and the test subject are mammals. In some embodiments of any of the above aspects, the control subject and the test subject are rodents, such as mice. In some embodiments of any of the above aspects, the control subject and the test subject are mice. In some embodiments of any of the above aspects, the control subject and the test subject are rats. In some embodiments of any of the above aspects, the control subject and the test subject are non-human primates. In some embodiments of any of the above aspects, the control subject and the test subject are domesticated animals, such as dogs, cats, sheep, cattle, horses, goats, pigs, mice, rats, rabbits, hamsters, and guinea pigs. In some embodiments of any of the above aspects, the control subject and the test subject are dogs. In some embodiments of any of the above aspects, the control subject and the test subject are cats. In some embodiments of any of the above aspects, the control subject and the test subject are sheep. In some embodiments of any of the above aspects, the control subject and the test subject are cattle. In some embodiments of any of the above aspects, the control subject and the test subject are horses. In some embodiments of any of the above aspects, the control subject and the test subject are goats. In some embodiments of any of the above aspects, the control subject and the test subject are pigs. In some embodiments of any of the above aspects, the control subject and the test subject are rabbits. In some embodiments of any of the above aspects, the control subject and the test subject are hamsters. In some embodiments of any of the above aspects, the control subject and the test subject are guinea pigs.

In some embodiments of any of the above aspects, the agent is administered to the test subject systemically or locally. In some embodiments of any of the above aspects, the agent is systemically administered to the test subject. In some embodiments of any of the above aspects, the agent is locally administered to the test subject. In some embodiments of any of the above aspects, the agent is administered to the test subject intravitreally, intravenously, intraperitoneally, orally, subcutaneously or intramuscularly. In some embodiments of any of the above aspects, the agent is administered to the test subject intravenously. In some embodiments of any of the above aspects, the agent is administered to the test subject intraperitoneally. In some embodiments of any of the above aspects, the agent is administered to the test subject orally. In some embodiments of any of the above aspects, the agent is administered to the test subject subcutaneously. In some embodiments of any of the above aspects, the agent is administered to the test subject intramuscularly. In some embodiments of any of the above aspects, the agent is administered to the test subject intravitreally. In some embodiments of any of the above aspects, the agent is administered to the test subject by intravitreal injection or through an intravitreal device. In some embodiments of any of the above aspects, the agent is administered to the test subject by intravitreal injection. In some embodiments of any of the above aspects, the agent is administered to the test subject through an intravitreal device.

In some embodiments of any of the above aspects, the agent is selected from the group consisting of a small molecule, an antibody, a polypeptide, a polynucleotide, and a gene therapy. In some embodiments of any of the above aspects, the agent is a small molecule. In some embodiments, the agent is an antibody. In some embodiments of any of the above aspects, the agent is a polypeptide. In some embodiments of any of the above aspects, the agent is a polynucleotide. In some embodiments of any of the above aspects, the agent is a gene therapy. In some embodiments of any of the above aspects, the agent is not a VEGF inhibitor. In some embodiments of any of the above aspect, the agent is not an anti-VEGF antibody.

In some embodiments of any of the above aspects, the control and test subjects are resistant or refractory to treatment with an anti-VEGF antibody.

Examples of VEGF inhibitors include, but are not limited to, AVASTIN® (bevacizumab), MACUGEN® (pegaptanib), EYLEA® (aflibercept), LUCENTIS® (ranibizumab), BEOVU® (brolucizumab), VABYSMO® (faricimab), LUMITIN® (conbercept), and biosimilars thereof.

### In Vitro Methods

The present disclosure further provides methods of modeling angiogenesis-associated diseases or conditions or wound healing in a subject, e.g., a subject that is resistant or refractory to treatment with a VEGF inhibitor, such as an anti-VEGF antibody. The methods utilize engineered cells described herein in which the Col8a1 gene has been knocked out or in which expression of Col8a1 gene has been knockdown and comprise the step of injuring the engineered cells.

In a fifth aspect, the present disclosure provides a method for determining the efficacy of an agent in modulating angiogenesis or promoting wound healing in injured cells comprising the steps of: (a) injuring a control culture of cells and a test culture of cells, wherein the Collagen Type VIII Alpha 1 Chain (Col8a1) gene in the cells has been knocked out or expression of the Col8a1 gene has been knocked down in both the control and test cell cultures, (b) contacting the cells of the test culture of cells with the agent, and (c) subsequently evaluating migration of cells across the injury and/or cell proliferation in the control culture of cells and the test culture of cells, wherein the agent is effective in modulating angiogenesis if there is a difference in migration of cells across the injury and/or a difference in cell proliferation between the test culture of cells and the control culture of cells, or wherein the agent is effective in promoting wound healing if cell migration across the injury and/or cell proliferation is faster in the test culture of cells than in the control culture of cells.

In some embodiments, step (c) comprises evaluating migration of cells across the injury in the control culture of cells and the test culture of cells, wherein the agent is effective in modulating angiogenesis if there is a difference in the rate of cell migration across the injury between the test culture of cells and the control culture of cells. In some embodiments, the agent is effective in modulating angiogenesis if there is at least 10% difference in the average rate of cell migration between the test cells and the control cells. In some embodiments, the agent is effective in modulating angiogenesis if there is at least 20% difference in the average rate of cell migration between the test cells and the control cells. In some embodiments, the agent is effective in modulating angiogenesis if there is at least 30% difference in the average rate of cell migration between the test cells and the control cells. In some embodiments, the agent is effective in modulating angiogenesis if there is at least 40% difference in the average rate of cell migration between the test cells and the control cells. In some embodiments, the agent is effective in modulating angiogenesis if there is at least 50% difference in the average rate of cell migration between the test cells and the control cells. In some embodiments, the agent is effective in modulating angiogenesis if there is at least 60% difference in the average rate of cell migration between the test cells and the control cells. In some embodiments, the agent is effective in modulating angiogenesis if there is at least 70% difference in the average rate of cell migration between the test cells and the control cells. In some embodiments, the agent is effective in modulating angiogenesis if there is at least 80% difference in the average rate of cell migration between the test cells and the control cells. In some embodiments, the agent is effective in modulating angiogenesis if there is at least 90% difference in the average rate of cell migration between the test cells and the control cells. In some embodiments, the agent is effective in modulating angiogenesis if there is at least 95% difference in the average rate of cell migration between the test cells and the control cells. In some embodiments, the agent is effective in modulating angiogenesis if there is at least 99% difference in the average rate of cell migration between the test cells and the control cells.

In some embodiments, step (c) comprises evaluating cell proliferation in the control culture of cells and the test culture of cells, wherein the agent is effective in modulating angiogenesis if there is a difference cell proliferation between the test culture of cells and the control culture of cells. In some embodiments, the agent is effective in modulating angiogenesis if there is at least 10% difference in the average rate of cell proliferation between the test cells and the control cells. In some embodiments, the agent is effective in modulating angiogenesis if there is at least 20% difference in the average rate of cell proliferation between the test cells and the control cells. In some embodiments, the agent is effective in modulating angiogenesis if there is at least 30% difference in the average rate of cell proliferation between the test cells and the control cells. In some embodiments, the agent is effective in modulating angiogenesis if there is at least 40% difference in the average rate of cell proliferation between the test cells and the control cells. In some embodiments, the agent is effective in modulating angiogenesis if there is at least 50% difference in the average rate of cell proliferation between the test cells and the control cells. In some embodiments, the agent is cell effective in modulating angiogenesis if there is at least 60% difference in the average rate of cell proliferation between the test cells and the control cells. In some embodiments, the agent is effective in modulating angiogenesis if there is at least 70% difference in the average rate of cell proliferation between the test cells and the control cells. In some embodiments, the agent is effective in modulating angiogenesis if there is at least 80% difference in the average rate of cell proliferation between the test cells and the control cells. In some embodiments, the agent is effective in modulating angiogenesis if there is at least 90% difference in the average rate of cell proliferation between the test cells and the control cells. In some embodiments, the agent is effective in modulating angiogenesis if there is at least 95% difference in the average rate of cell proliferation between the test cells and the control cells. In some embodiments, the agent is effective in modulating angiogenesis if there is at least 99% difference in the average rate of cell proliferation between the test cells and the control cells. In some embodiments, the agent is effective in modulating angiogenesis if there is at least 10% difference in the average rate of cell proliferation between the test cells and the control cells.

In some embodiments, step (c) comprises evaluating migration of cells across the injured cells in the control culture of cells and the test culture of cells, wherein the agent is effective in promoting wound healing if the migration of cells across the injury is faster in the test culture of cells than in the control culture of cells. In some embodiments, the agent is effective in promoting wound healing if the average rate of migration of the test cells is at least 10% faster than the average rate of migration of the control cells. In some embodiments, the agent is effective in promoting wound healing if the average rate of migration of the test cells is at least 20% faster than the average rate of migration of the control cells. In some embodiments, the agent is effective in promoting wound healing if the average rate of migration of the test cells is at least 30% faster than the average rate of migration of the control cells. In some embodiments, the agent is effective in promoting wound healing if the average rate of migration of the test cells is at least 40% faster than the average rate of migration of the control cells. In some embodiments, the agent is effective in promoting wound healing if the average rate of migration of the test cells is at least 50% faster than the average rate of migration of the control cells. In some embodiments, the agent is effective in promoting wound healing if the average rate of migration of the test cells is at least 60% faster than the average rate of migration of the control cells. In some embodiments, the agent is effective in promoting wound healing if the average rate of migration of the test cells is at least 70% faster than the average rate of migration of the control cells. In some embodiments, the agent is effective in promoting wound healing if the average rate of migration of the test cells is at least 80% faster than the average rate of migration of the control cells. In some embodiments, the agent is effective in promoting wound healing if the average rate of migration of the test cells is at least 90% faster than the average rate of migration of the control cells. In some embodiments, the agent is effective in promoting wound healing if the average rate of migration of the test cells is at least 95% faster than the average rate of migration of the control cells. In some embodiments, the agent is effective in promoting wound healing if the average rate of migration of the test cells is at least 99% faster than the average rate of migration of the control cells.

In some embodiments, step (c) comprises evaluating cell proliferation in the control culture of cells and the test culture of cells, wherein the agent is effective in promoting wound healing if the cell proliferation is faster in the test culture of cells than in the control culture of cells. In some embodiments, the agent is effective in promoting wound healing if the average rate of proliferation of the test cells is at least 10% faster than the average rate of proliferation of the control cells. In some embodiments, the agent is effective in promoting wound healing if the average rate of proliferation of the test cells is at least 20% faster than the average rate of proliferation of the control cells. In some embodiments, the agent is effective in promoting wound healing if the average rate of proliferation of the test cells is at least 30% faster than the average rate of proliferation of the control cells. In some embodiments, the agent is effective in promoting wound healing if the average rate of proliferation of the test cells is at least 40% faster than the average rate of proliferation of the control cells. In some embodiments, the agent is effective in promoting wound healing if the average rate of proliferation of the test cells is at least 50% faster than the average rate of proliferation of the control cells. In some embodiments, the agent is effective in promoting wound healing if the average rate of proliferation of the test cells is at least 60% faster than the average rate of proliferation of the control cells. In some embodiments, the agent is effective in promoting wound healing if the average rate of proliferation of the test cells is at least 70% faster than the average rate of proliferation of the control cells. In some embodiments, the agent is effective in promoting wound healing if the average rate of proliferation of the test cells is at least 80% faster than the average rate of proliferation of the control cells. In some embodiments, the agent is effective in promoting wound healing if the average rate of proliferation of the test cells is at least 90% faster than the average rate of proliferation of the control cells. In some embodiments, the agent is effective in promoting wound healing if the average rate of proliferation of the test cells is at least 95% faster than the average rate of proliferation of the control cells. In some embodiments, the agent is effective in promoting wound healing if the average rate of proliferation of the test cells is at least 99% faster than the average rate of proliferation of the control cells.

In some embodiments, the method is for determining the efficacy of an agent in promoting angiogenesis in injured cells. In some embodiments, the control and test cells are endothelial cells, fibroblasts or retinal pigment epithelial (RPE) cells. In some embodiments, the control and test cells are endothelial cells. In some embodiments, the control and test cells are vascular endothelial cells (vECs). In some embodiments, the control and test cells are fibroblasts. In some embodiments, the control and test cells are retinal pigment epithelial (RPE) cells.

In some embodiments, the cells are any of the engineered cells disclosed herein. In some embodiments, the cells are human cells. In some embodiments, the cells are differentiated from pluripotent stem cells (PSCs). In some embodiments, the cells are differentiated from embryonic stem cells (ESCs). In some embodiments, the cells are differentiated from induced pluripotent stem cells (iPSCs). In some embodiments, the cells are differentiated from human embryonic stem cells (hESCs). In some embodiments, the cells are vascular endothelial cells.

In some embodiments, the method comprises knocking out the Col8a1 gene in a (pluripotent stem cell) PSC, e.g., an embryonic stem cells (ESC) or an induced pluripotent stem cells (iPSC), and differentiating the Col8a1 knockout PSC into a vascular endothelial cell prior to said injuring. In some embodiments, the Col8a1 gene is knocked out from the PSC using CRISPR/Cas technology.

In some embodiments, the injuring is a scratch. In some embodiments, the contacting step comprises adding the agent to the culture medium of the test culture of cells after the test culture of cells is injured. In some embodiments, the contacting step comprises adding the agent to the culture medium of the test culture of cells before the test culture of cells is injured.

In some embodiments, the agent is selected from the group consisting of a small molecule, an antibody, a polypeptide, a polynucleotide, and a gene therapy. In some embodiments of any of the above aspects, the agent is a small molecule. In some embodiments, the agent is an antibody. In some embodiments of any of the above aspects, the agent is a polypeptide. In some embodiments of any of the above aspects, the agent is a polynucleotide. In some embodiments of any of the above aspects, the agent is a gene therapy. In some embodiments, the agent is not a VEGF inhibitor. In some embodiments of any of the above aspect, the agent is not an anti-VEGF antibody.

In some embodiments, the method further comprises contacting the cells of the test culture and the control culture with a VEGF inhibitor, such as an anti-VEGF antibody. In some embodiments, the cells of the test culture are contacted with the VEGF inhibitor prior to said contacting with the agent. In some embodiments, the cells of the test culture are simultaneously contacted with the VEGF inhibitor and the agent.

Examples of VEGF inhibitors include, but are not limited to, AVASTIN® (bevacizumab), MACUGEN® (pegaptanib), EYLEA® (aflibercept), LUCENTIS® (ranibizumab), BEOVU® (brolucizumab), VABYSMO® (faricimab), LUMITIN® (conbercept), and biosimilars thereof.

### EXAMPLES

### Example 1: Generation of Collagen Type VIII Alpha 1 Chain Knockout Mouse by CRISPR

Collagen Type VIII Alpha 1 Chain knockout (Col8a1 ko/ko) mice were obtained by electroporation-based strategy of C57BL/6J zygotes with 25 ng/μL wild-type Cas9 mRNA (Life Technologies) and 13 ng/μL of two in vitro-transcribed single-guide RNA (sgRNA) into mouse zygotes (Modzelewski et al. (2018) Nat Protoc. 13: 1253-1274). Target sequences of sgRNA used to knockout exon 4 are listed in Table 1. The 2150 bp knockout region corresponds to GRCm38/mm10 chr16: 57,631,573-57,633,722.

TABLE 1
Oligonucleotide sequences for sgRNA
Sequences for COL8A1 knockout (KO) animals

| Oligo | | | CFD |
|---|---|---|---|
| Name | Sequence | PAM¹ | Score² |
| Col8a1 | 5′-GTAGGAGCTACGTCAAAATT-3′ | TGG | 97 |
| | (SEQ ID NO: 1) | | |
| Col8a1 | 5′-CAGTTAAGAGTGTTCGGCAC-3′ | TGG | 97 |
| | (SEQ ID NO: 2) | | |

¹The “PAM” or “protospacer adjacent motif” refers to a short DNA sequence (approximately 2-6 base pairs in length) that follows the DNA region targeted for cleavage by the CRISPR system. The PAM is required for a Cas nuclease to cut and is generally found 3-4 nucleotides downstream from the cut site.
²The “CFD Score” or “cutting frequency determination score” refers to the measure of specificity of a guide RNA to the target DNA. The CFD score ranges between 0 and 100 for each guide, with 100 being the strongest interaction between the guide and the target and 0 being the weakest interaction due to mismatches between the guide RNA and the DNA target.

Tail DNA from resulting offspring was analyzed by PCR and sequencing. Genotyping was carried out using the primers listed in Table 2. The band size for the wild-type allele was 401 bp and 259 bp for the knockout allele.

TABLE 2
PCR Primers for
Genotyping COL8A1 knockout (KO) animals

| | Sequence |
|---|---|
| Col8a1ko 1: | TTGTACTAGATGCTCCTTCCT |
| | (SEQ ID NO: 3) |
| Col8a1ko 2: | GCTTCCTGATGGTTTGGT |
| | (SEQ ID NO: 4) |
| Col8a1ko 3: | TTGGTATTGCTGTTACACTTATTG |
| | (SEQ ID NO: 5) |

The sequence of the mouse COL8A1 protein is disclosed below, which corresponds to Gene bank Accession NO. NP_031765.2. The bolded/underlined amino acids (G110 to R130) have been deleted in the CRISPR knockout.

(SEQ ID NO: 6)
MAVPPRPLQLLGILFIISLNSVRLIQAGAYYGIKPLPPQIPPQIPPQIP
QYQPLGQQVPHMPLGKDGLSMGKEMPHMQYGKEYPHLPQYMKEIPPVPR
MGKEVVPKKGKGEVPLASLRGEQGPRGEPGPRGPPGPPGLPGHGMPGIK
GKPGPQGYPGIGKPGMPGMPGKPGAMGMPGAKGEIGPKGEIGPMGIPGP
QGPPGPHGLPGIGKPGGPGLPGQPGAKGERGPKGPPGPPGLQGPKGEKG
FGMPGLPGLKGPPGMHGPPGPVGLPGVGKPGVTGFPGPQGPLGKPGPPG
EPGPQGLIGVPGVQGPPGMPGVGKPGQDGIPGQPGFPGGKGEQGLPGLP
GPPGLPGVGKPGFPGPKGDRGIGGVPGVLGPRGEKGPIGAPGMGGPPGE
PGLPGIPGPMGPPGAIGFPGPKGEGGVVGPQGPPGPKGEPGLQGFPGKP
GFLGEVGPPGMRGLPGPIGPKGEGGHKGLPGLPGVPGLLGPKGEPGIPG
DQGLQGPPGIPGIVGPSGPIGPPGIPGPKGEPGLPGPPGFPGVGKPGVA
GLHGPPGKPGALGPQGQPGLPGPPGPPGPPGPPAVMPTPSPQGEYLPDM
GLGIDGVKPPHAYAGKKGKHGGPAYEMPAFTAELTVPFPPVGAPVKFDK
LLYNGRQNYNPQTGIFTCEVPGVYYFAYHVHCKGGNVWVALFKNNEPMM
YTYDEYKKGFLDQASGSAVLLLRPGDQVFLQMPSEQAAGLYAGQYVHSS
FSGYLLYPM

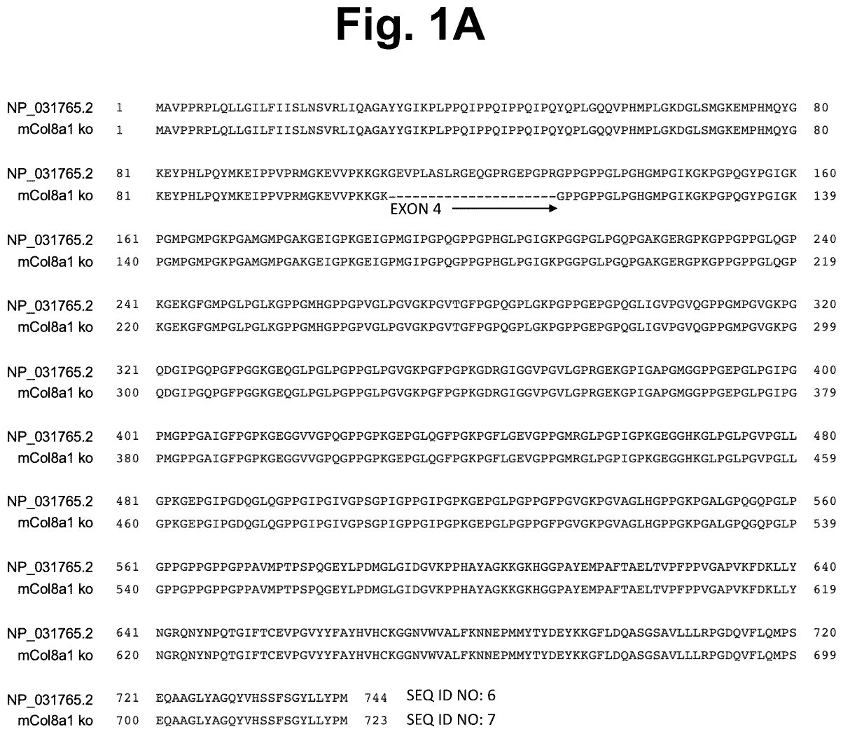

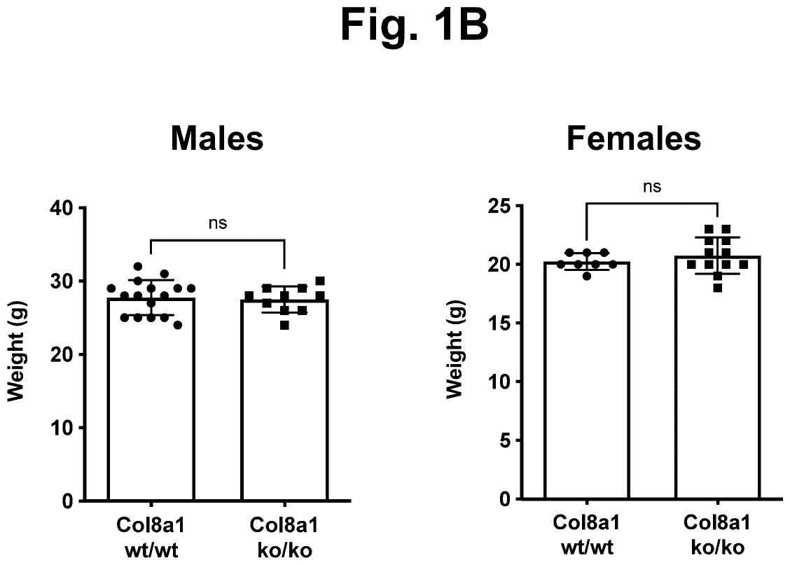

The protein sequence alignment (FIG. 1A) and DNA sequence alignment (data not shown) indicate the deletion at exon 4 in the Col8a1 knockout mice. Compared to Col8a1 wildtype littermates, Col8a1 knockout mice were found to be healthy and have similar weights between ages 2.5 to 5 months (FIG. 1B and Table 3, infra).

TABLE 3
Weight Comparison of COL8A1 knockout and wildtype littermates

| | Males | | Females | |
|---|---|---|---|---|
| | Col8a1 wt/wt | Col8a1 ko/ko | Col8a1 wt/wt | Col8a1 ko/ko |
| Mean Weight (g) | 27.75 | 27.50 | 20.25 | 20.75 |
| Standard Deviation (g) | 2.38 | 1.78 | 0.70 | 1.54 |
| Unpaired t-test: p- | 0.7780 | | 0.4054 | |
| value | | | | |

### Example 2: Laser-Induced Choroidal Neovascularization (CNV) Mouse Model

Mice were anesthetized by intraperitoneal injection of ketamine (70-80 mg/kg body weight) and xylazine (15 mg/kg body weight). Pupils were dilated with drops of Tropicamide Ophthalmic Solution USP 1% (Akorn). Drops of Systane lubricant eye drop (Alcon) were applied bilaterally to prevent corneal dehydration during the procedure. Mice also received analgesic (buprenorphine, 0.05 mg/kg) by intraperitoneal injection the day of the procedure. Choroidal neovascularization was induced in each eye using an image-guided Optical Coherence Tomography (OCT) system (Micron III, Phoenix Research Laboratories, Pleasanton, CA) with a laser spot size of 100 m, 320 mW power and 80 ms duration. Laser burns aiming at the Brusch's membrane between the choroid and the retinal pigment epithelium (RPE) were performed. Four burns at the 0, 3, 6, and 9 o'clock positions around the optic nerve at approximately 2-3 optic disk diameters (about 200-300 m) from the optic nerve in both the left and right eye were made. The presence of a bubble at the time of laser application confirmed sufficient rupture of Bruch's membrane. Cases of subretinal hemorrhage induced by the laser were excluded from the analysis.

After the laser procedure, the eyes were treated with an antibiotic ointment (Neomycin and Polymyxin B Sulfates and Bacitracin Zinc Ophthalmic Ointment, Bausch & Lomb). Mice were then placed on a pre-warmed warming plate at 37° C. until they awakened. After 7 days, mice received an intravenous injection of 0.1 mg FITC-lectin (Lycopersicon esculentum FL1171, Vector Labs) and five minutes later were euthanized with C02. Eyes were enucleated and either fixed in Davidson's fixative solution for 24 h before formalin fixation and paraffin embedding (FFPE), sectioning and Hematoxylin Eosin (H&E) staining or fixed in 10% Neutral Buffered Formalin (NBF) for 1 h before dissection and flatmounting of the choroid/RPE samples. CNV lesion size quantification was performed using fluorescence microscopy.

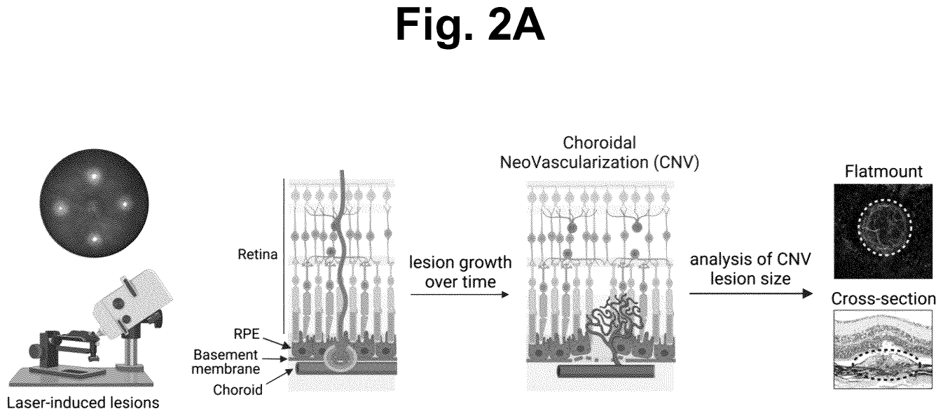

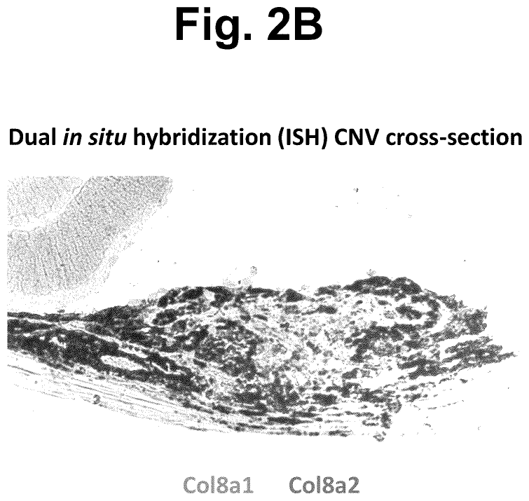

FIG. 2A provides an overview of the laser-induced choroidal neovascularization (CNV) mouse pre-clinical model. Dual in situ hybridization (ISH) of Col8a1 and Col8a2 mRNA confirmed high levels of Col8a1 expression (green) in CNV lesions and low level Col8a2 expression in the RPE and choroid surrounding the lesions as observed in the human AMD lesions (FIG. 2B).

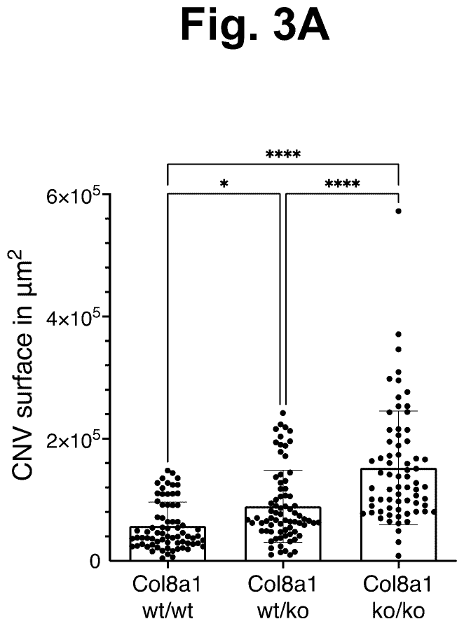

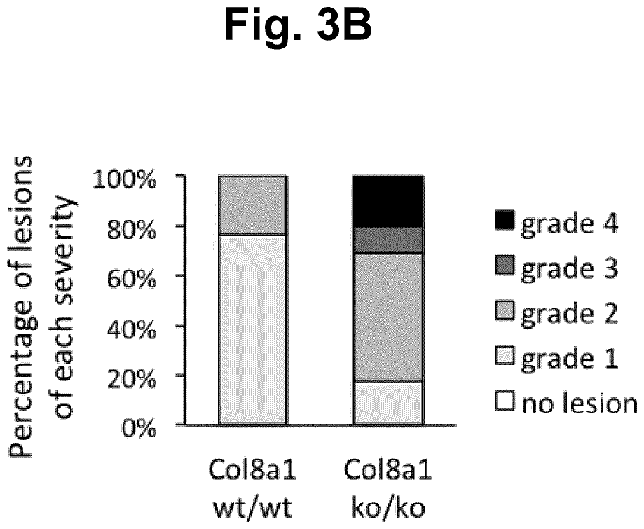

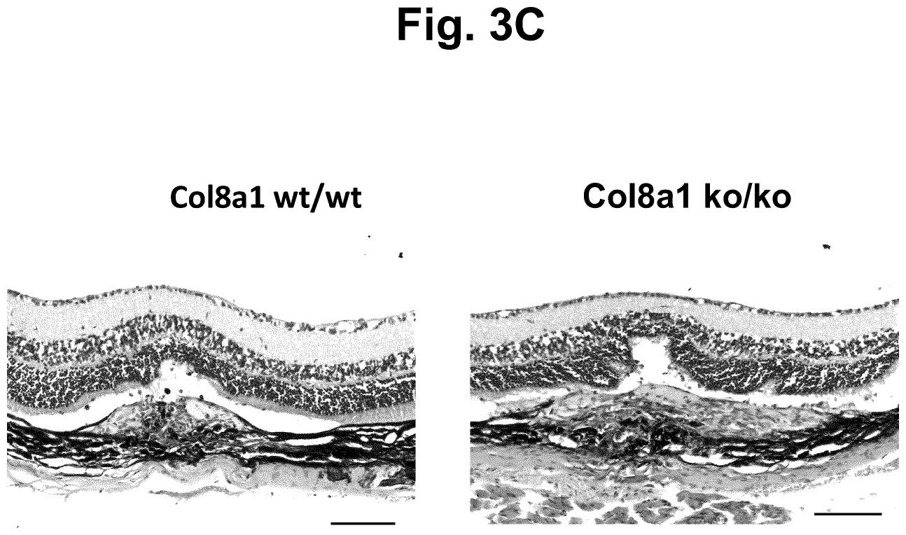

At 7 days post induction of CNV, Col8a1 knockout mice were found to have an increase in CNV lesion surface size compared to Col8a1 wildtype and heterozygote mice (FIGS. 3A and 3C, and Table 4, infra). Col8a1 knockout mice also experienced a greater severity of lesions. Approximately 76.7% of resulting CNV lesions in Col8a1 wildtype mice were grade 1 lesions (<0.9 μm²) with the remaining 23.3% being grade 2 lesions (<1.7 μm²). About 17.8% of lesions in Col8a1 knockout mice were categorized as grade 1, while approximately 51.1% were grade 2, 11.1% were grade 3 (<2.5 μm²) and 20% were grade 4 (<3.3 μm²) (FIG. 3B and Table 5, infra). H&E staining revealed an increase in infiltrating cells into the CNV lesions in Col8a1 knockout mice compared to controls. Overall, these data show that Col8a1 knockout exacerbated laser-induced CNV lesion size at Day 7.

TABLE 4
CNV Lesion Size in COL8A1 knockout,
heterozygous and wildtype mice

| | Col8a1 wt/wt | Col8a1 wt/ko | Col8a1 ko/ko |
|---|---|---|---|
| CNV Mean Surface | 57,043 | 89,218 | 152,213 |
| Area (μm²) | | | |
| Standard Deviation | 39,034 | 59,291 | 93,134 |
| (μm²) | | | |

TABLE 5
CNV Lesion Severity in COL8A1 knockout and wildtype mice

| % Lesions of each severity | Col8a1 wt/wt | Col8a1 ko/ko |
|---|---|---|
| No Lesion | 0.0 | 0.0 |
| Grade 1 | 76.7 | 17.8 |
| Grade 2 | 23.3 | 51.1 |
| Grade 3 | 0.0 | 11.1 |
| Grade 4 | 0.0 | 20.0 |

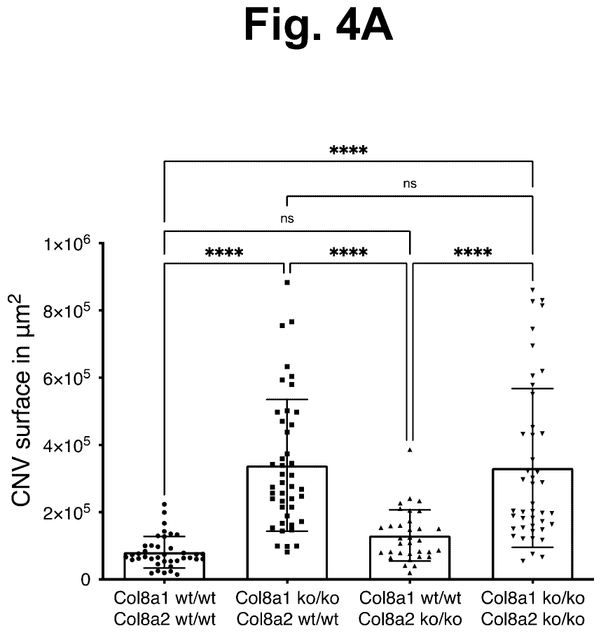

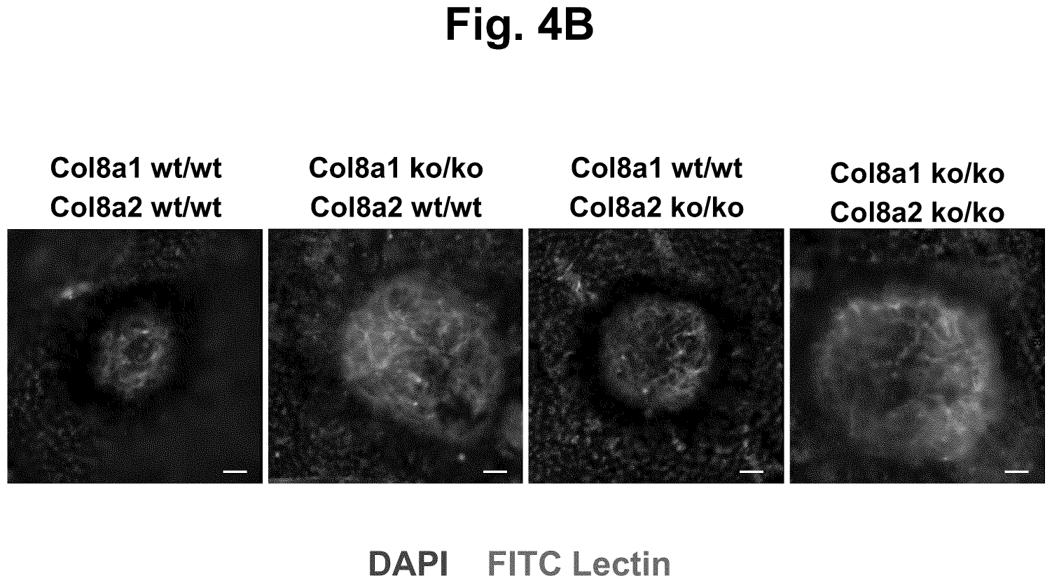

To investigate whether both Col8a1 and Col8a2 affected CNV lesion formation, laser-induced CNV lesions were compared among wild type (Col8a1 wt/wt; Col8a2 wt/wt), Col8a1 knockout (Col8a1 ko/ko; Col8a2 wt/wt), Col8a2 knockout (Col8a1 wt/wt; Col8a2 ko/ko), and Col8a1; Col8a2 double knockout (Col8a1 ko/ko; Col8a2 ko/ko) mice at Day 7 post induction. While Col8a2 knockout mice experienced CNV lesion of similar size to wildtype mice, Col8a1 knockout and Col8a1; Col8a2 double knockout mice showed a significant increase in CNV lesion size (FIG. 4A and Table 6). Immunofluorescence analysis showed an increase in neovascularization, as indicated by an increase in FITC-Lectin staining, in Col8a1 knockout and Col8a1; Col8a2 double knockout mice compared to wildtype and Col8a2 knockout mice (FIG. 4B), confirming that the exacerbation in laser-induced CNV lesion size is caused by loss of Col8a1 and not Col8a2.

TABLE 6
CNV Lesion Size in COL8A1 knockout
and COL8A2 knockout mice

| | Col8a1 | Col8a1 | Col8a1 | Col8a1 |
|---|---|---|---|---|
| | wt/wt | ko/ko | wt/wt | ko/ko |
| | Col8a2 | Col8a2 | Col8a2 | Col8a2 |
| | wt/wt | wt/wt | ko/ko | ko/ko |
| CNV Mean Surface | 81,105 | 339,069 | 130,763 | 331,549 |
| Area (μm²) | | | | |
| Standard Deviation | 47,006 | 195,815 | 75,863 | 235,901 |
| (μm²) | | | | |

Laser-Induced CNV and Treatment with Anti-VEGF Antibody

The effect of anti-VEGF antibody treatment on laser induced CNV lesion formation was investigated by treating wildtype and Col8a1 knockout mice with an anti-VEGF antibody and anti-gp120 antibody as a control. Laser-induced CNV lesions were made in Col8a1 wildtype and knockout mice using the above procedure on Day 0. The mice were treated with 5 mg/kg anti-VEGF antibody B20 (Liang W C et al., J. Biol. Chem. 2006; 281(2): 951-61) or 5 mg/kg anti-gp20 antibody via intraperitoneal (i.p.) injection on Days 0, 1, 2, 3, 4 and 5. Mice were euthanized on Day 7 and the size of CNV lesions were measured and compared. Experiments in which mice were treated with 5 mg/kg anti-VEGF antibody i.p. injection for three times on Days 0, 2 and 5, or 0, 3 and 5 led to similar results.

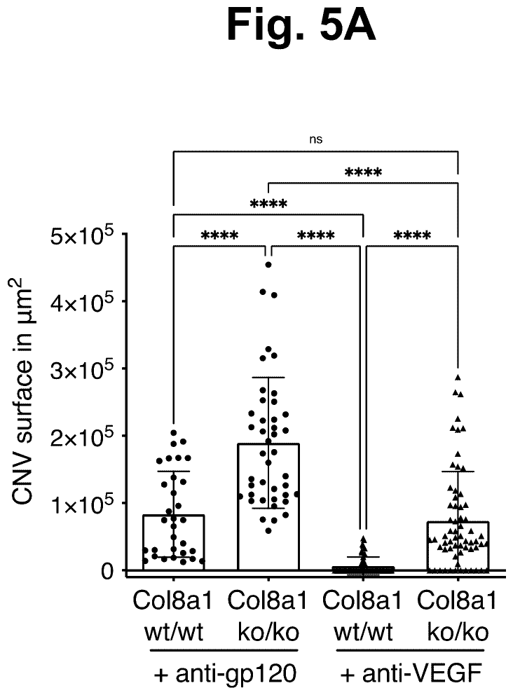

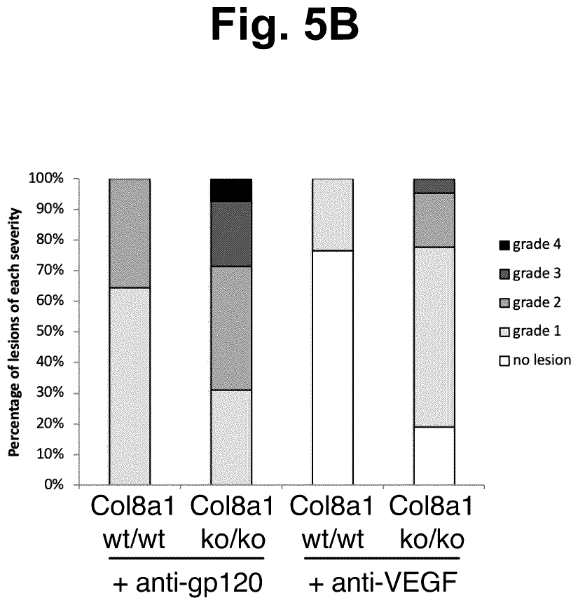

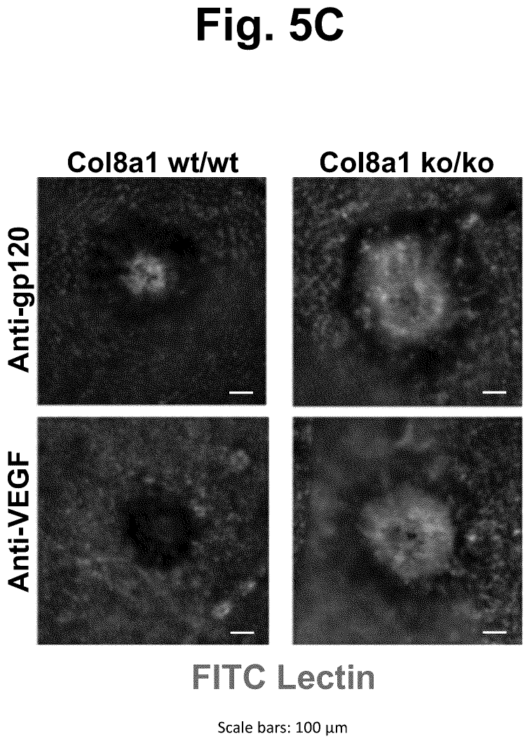

Following anti-VEGF antibody treatment, Col8a1 wildtype animals showed a significant decrease in lesion size and severity (FIGS. 5A-5C and Tables 7 and 8). Approximately 76.5% of lesions in wildtype mice were fully resolved following anti-VEGF antibody treatment. Comparatively, Col8a1 knockout animals showed very poor response to anti-VEGF antibody treatment, with only about 19% of Col8a1 knockout lesions being resolved (FIG. 5B and Table 8). Immunofluorescence analysis also showed a decrease in neovascularization, indicated by a decrease in FITC-Lectin vascular staining, in wildtype animals treated with anti-VEGF antibody compared to the wildtype animals treated with anti-gp120 antibody and the Col8a1 knockout mice in both treatment groups. Col8a1 knockout mice treated with anti-VEGF antibody showed similar levels of neovascularization compared to the Col8a1 knockout anti-gp120 antibody-treated group. (FIG. 5C).

TABLE 7
CNV Lesion Size in COL8A1 knockout
and COL8A2 knockout mice

| | +anti-gp120 | | +anti-VEGF | |
|---|---|---|---|---|
| | Col8a1 | Col8a1 | Col8a1 | Col8a1 |
| | wt/wt | ko/ko | wt/wt | ko/ko |
| CNV Mean Surface | 83,333 | 189,465 | 6,631 | 73,321 |
| Area (μm²) | | | | |
| Standard Deviation | 63,963 | 97,251 | 13,421 | 73,439 |
| (μm²) | | | | |

TABLE 8
CNV Lesion Severity in COL8A1 knockout and wildtype
mice in response to anti-VEGF treatment

| | |
|---|---|
| +anti-gp120 | +anti-VEGF |

| % Lesions of | Col8a1 | Col8a1 | Col8a1 | Col8a1 |
|---|---|---|---|---|
| each severity | wt/wt | ko/ko | wt/wt | ko/ko |
| No Lesion | 0.0 | 0.0 | 76.5 | 19.0 |
| Grade 1 | 64.5 | 31.0 | 23.5 | 58.7 |
| Grade 2 | 35.5 | 40.5 | 0.0 | 17.5 |
| Grade 3 | 0.0 | 21.4 | 0.0 | 4.8 |
| Grade 4 | 0.0 | 7.1 | 0.0 | 0.0 |

### Example 3: Sodium Iodate Mouse Model

Female mice were intravenously injected with 20 mg/kg body weight of sodium iodate (NaIO₃, Sigma-Aldrich) or saline control. For Optical Coherence Tomography (OCT), mice were anesthetized by intraperitoneal injection of ketamine (70-80 mg/kg body weight) and xylazine (15 mg/kg body weight). Pupils were dilated with drops of Tropicamide Ophthalmic Solution USP 1% (Akorn). Drops of Systane lubricant eye drop (Alcon) were applied bilaterally to prevent corneal dehydration during the procedure. OCT retinal scans were recorded using a Bioptigen Envisu R machine (Leica Microsystems, IL, USA). After ocular examination, anesthetized mice were placed on a pre-warmed warming plate at 37° C. until they awakened. For histology, eyes were enucleated 21 days post NaIO₃ injection and fixed in Davidson's fixative solution for 24 h before FFPE, sectioning and Hematoxylin Eosin staining.

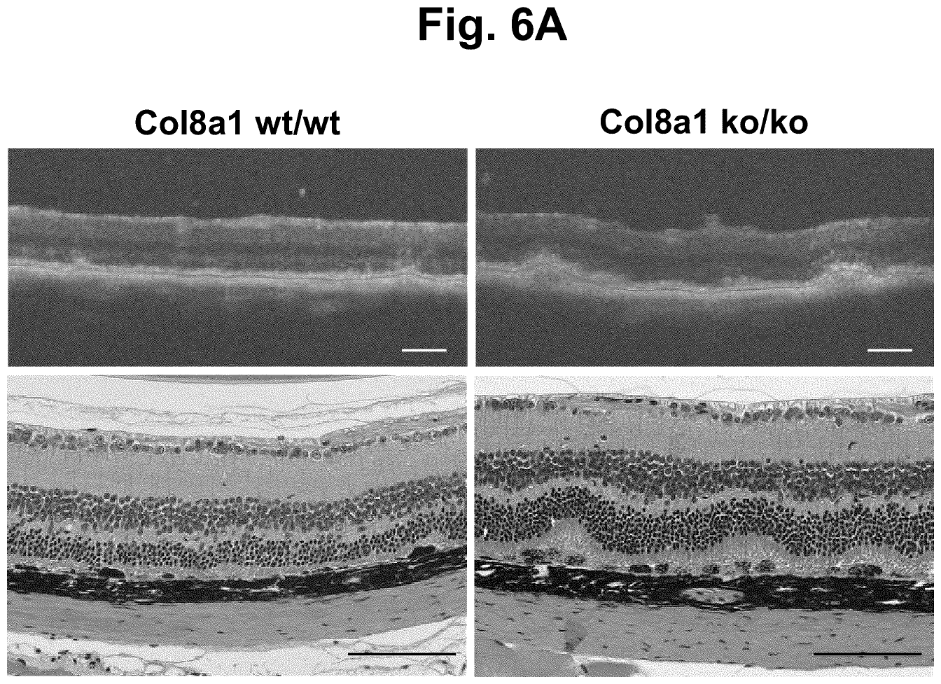

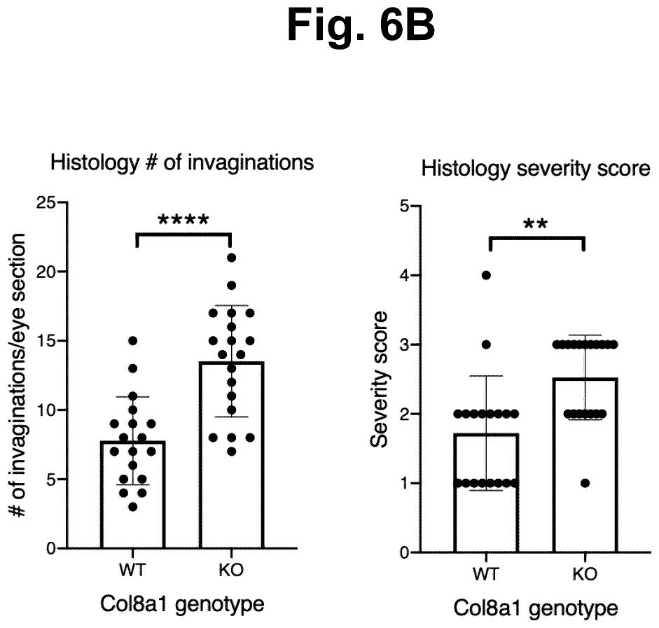

Loss of Col8a1 resulted in larger subretinal lesions in the sodium iodate mouse model of AMD. Optical coherence tomography (OCT) and histological analysis demonstrated that Col8a1 knockout animals showed greater disorganization of retinal layers when compared to Col8a1 wildtype littermates (FIG. 6A). Col8a1 knockout animals were found to have disproportionately more severe injury to the RPE than wildtype animals with a similar degree of cell loss in the outer nuclear layer (ONL). These more severe lesions were characterized by nodular aggregates of plump spindle cells, many of which contained melanin. The larger aggregates bulged into the overlying photoreceptor layer and resulted in wave-like folding of the retina (FIG. 6A, bottom panels). This was reflected by an increase in the number of retinal invaginations and greater severity of lesions in Col8a1 knockout animals than Col8a1 wildtype controls at 21 days post NaIO₃ injection (FIG. 6B).

### Example 4: In Vitro Functional Assays

Generation of Collagen Type VIII Alpha 1 Chain Knockout Human Embryonic Stem Cell (hESC)

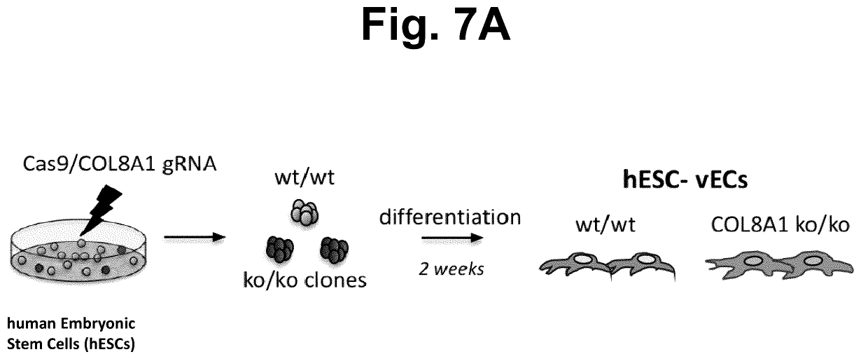

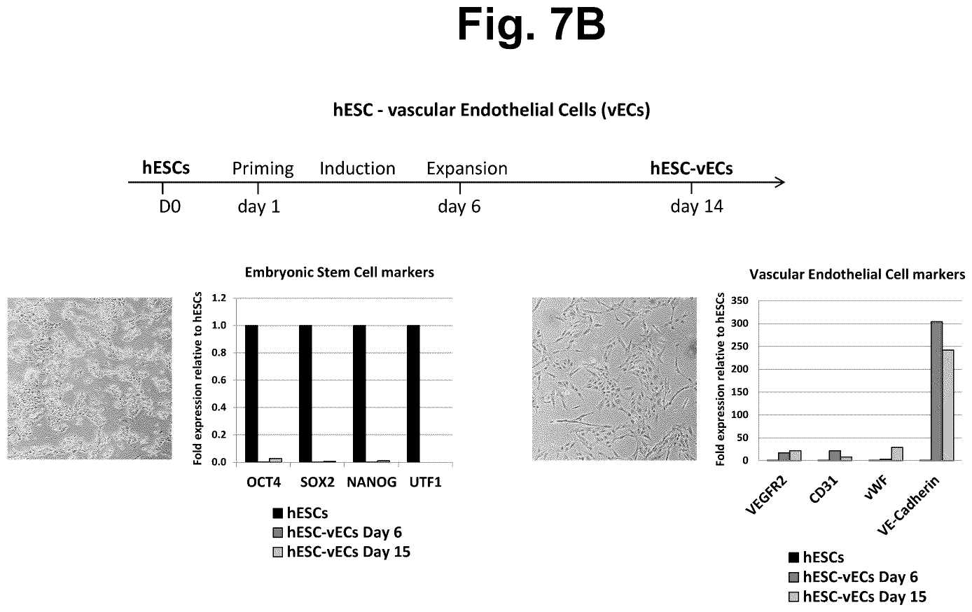

Targeted deletion of Collagen Type VIII Alpha 1 Chain (Col8a1) in human embryonic stem cells (hESCs) was accomplished by CRISPR-Cas9-based knockout through a 2-cut strategy (FIG. 7A). Under this strategy, a synthetic single-guide RNA (sgRNA) recognizes a DNA sequence and guides a cut by Cas9 protein in the coding exon 4 of COL8A1 while the other recognizes and introduces a cut in the intron 4. This strategy can disrupt the coding frame and minimize the chance of generating in-frame truncated protein.

Specifically, 180 picomolar (pM) of each sgRNA listed in Table 9 (synthesized by Synthego) were pre-complexed with 60 pM recombinant Cas9 protein v2 (ThermoFisher Scientific A36499) in a final volume of 6 microliters (μL) and incubated at room temperature for 10 min to form a ribonucleoprotein (RNP) complex immediately before electroporation. Each RNP complex was mixed with 200,000 hESCs (passage 24) in 20 μL of supplemented P3 buffer (Lonza), transferred to a Nucleocuvette strip (Lonza), and electroporated using Nucleofector X-Unit and a preset program CB-150 (Lonza). Once the nucleofection was completed, the Nucleocuvette was removed and 80 μL prewarmed supplemented mTeSR plus media (STEMCELL technologies 100-0274) with a final concentration of 10 micromolar (μM) ROCK inhibitor Y-27632 (STEMCELL technologies 72304) was added to mix with each reaction. 50 μL of the reaction was transferred to a well in a 12-well culture plate pre-coated with Matrigel (Corning 40234) and 50 μL of the reaction was transferred to a well in a Matrigel coated 6-well culture plate. After 24 hours, the cells were changed to regular mTeSR plus media without ROCK inhibitor and cultured for 3-7 days.

Bulk genomic DNAs (gDNAs) were collected from 12-well plates using a Quick-DNA microprep kit (Zymo Research D3020) following the manufacturer's instruction. Targeted PCR was performed to determine the deletion of Col8a1 at the bulk DNA level using the primers listed in Table 9.

TABLE 9
Oligonucleotide sequences for sgRNA
Sequences and PCR Primers for
Genotyping COL8A1 knockout (KO) cells

| Oligo Name | Sequence |
|---|---|
| sgRNA1 | 5′-GAGTTCCATCAGGCTCATCC-3′ |
| | (SEQ ID NO: 8) |
| sgRNA2 | 5′-TTGCGAACTAAGTATTAGAC-3′ |
| | (SEQ ID NO: 9) |
| Forward: | 5′-GGTTCCTCCCACAGGTGATG-3′ |
| | (SEQ ID NO: 10) |
| Reverse | 5′-TGTCACACTGAGGGGCATTTTGTTGGT-3′ |
| | (SEQ ID NO: 11) |

To identify a homozygous Col8a1 knockout (Col8a1 ko/ko)) clone, an individual colony was first carefully picked from a 6-well using a P200 pipette onto each of the matrigel-coated 48-well plate in a final volume of 250 μL supplemented mTeSR plus media with 10 μM ROCK inhibitor Y-27632. After 24 hours, the cells were changed to regular mTeSR plus media without ROCK inhibitor and cultured until the colony had grown sufficiently. Next, approximately half of the individual colony was collected for gDNA and PCR, and the other half was seeded onto a new Matrigel-coated 48-well plate to allow growth. TA cloning and Sanger sequencing were performed to examine the allele-specific deletion to ensure the generation of the homozygous Col8a1 knockout clone. Once confirmed, the cells were expanded and cryopreserved.

Directed Differentiation of the Human Embryonic Stem Cells (hESCs) into Vascular Endothelial Cells (vEC)

Col8a1 wildtype (Col8a1 wt/wt) and Col8a1 knockout (Col8a1 ko/ko) hESCs of similar passages (between passage 30-40) were seeded onto any size flask at a density of 37,000-47,000 cells per cm² in supplemented mTeSR plus media with 10 μM ROCK inhibitor Y-27632 and incubated at 37° C. After 24 hours, mTeSR medium was replaced with Priming Medium with a final concentration of 8 μM CHIR-99021 (STEMCELL technologies 72054) and 25 ng/mL recombinant human BMP4 (Peprotech 120-05) for 3 days without medium change. The composition of the Priming Medium in a 200 mL format included 94 mL DMEM:F12 medium (ThermoFisher Scientific 11320033), 100 mL Neurobasal (ThermoFisher Scientific 21103049), 4 mL 50×B27 supplement minus insulin (ThermoFisher Scientific 12587010), 2 mL 100×N2 supplement (ThermoFisher Scientific 17502048) and 200 μL 50 mM 2-Mercaptoethanol (ThermoFisher Scientific 31350010). After 3 days, the cells were replenished daily with the supplemented StemPro-34 medium (ThermoFisher Scientific 10639011) with a final concentration of 500 ng/mL recombinant human VEGF165 (Peprotech 100-20) and 2 μM Forskolin (STEMCELL technologies 72114) for two days. After 2 days, the cells were dissociated and seeded at a density of 25,000 cells per cm² onto any size flask coated with 25 μg/mL fibronectin (Corning 356008)—passage 1 of differentiated vascular endothelial cells (vECs). Purity of the vECs was confirmed by quantitative PCR of characteristic embryonic stem cell markers (OCT4, SOX2, NANOG and UTF1) and endothelial markers (VEGFR2, CD31, vWF and VE-Cadherin). See FIG. 7B.

### Migration and Proliferation Assay

Differentiated Col8a1 wildtype (Col8a1 wt/wt) and Col8a1 knockout (Col8a1 ko/ko) vECs were seeded at a density of 300,000 cells per mL in 100 μL supplemented StemPro-34 medium (ThermoFisher Scientific 10639011) with a final concentration of 500 ng/mL recombinant human VEGF165 (Peprotech 100-20) into each well of a 96-well ImageLock tissue culture plate (Sartorius BA-04857) and incubated in a standard cell incubator for between 6-18 hours. The plates were then removed from the incubator and precise and uniform wounds were created by Incucyte WoundMaker tool (Sartorius 4563) in all wells of the 96-well ImageLock tissue culture plate according to manufacturer's instructions. Each well was gently washed twice with culture media after wounding to prevent dislodged cells from settling and reattaching. Next, 100 μL of culture media containing test material at appropriate concentrations (e.g., anti-gp120 and anti-VEGF) was added to each well. The 96-well ImageLock tissue culture plates were then placed into the IncuCyte S3 Live Cell Analysis Instrument and imaged every 2-3 hours for 96 hours using the IncuCyte Scratch Wound Analysis Software Module.

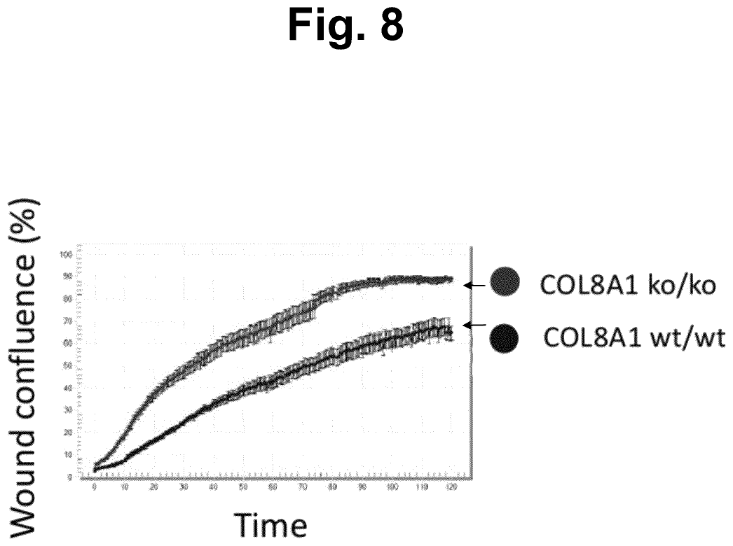

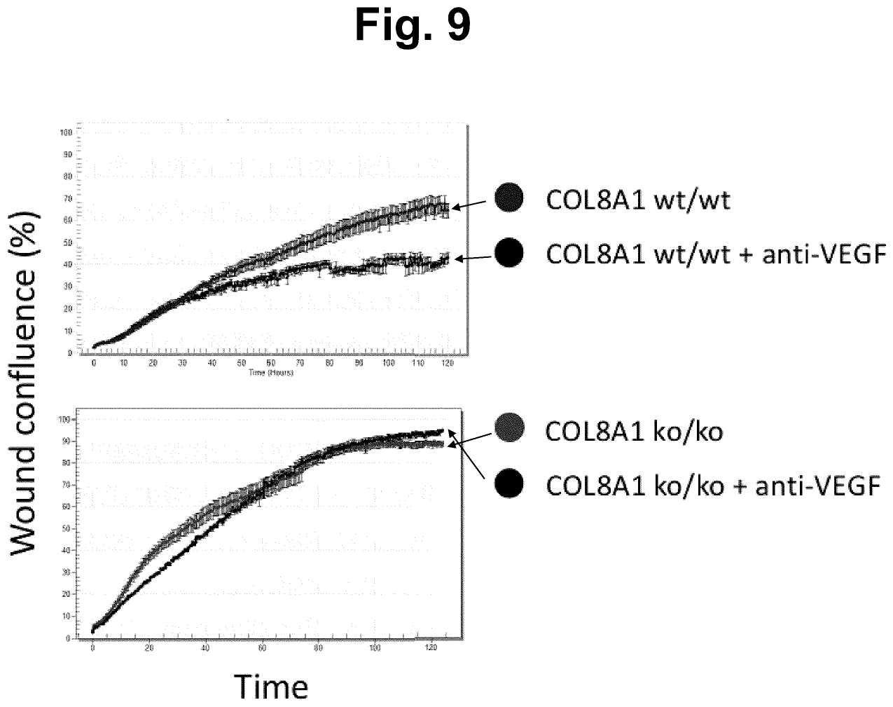

Col8a1 knockout hESC-derived vECS showed increased migration and proliferation when compared to Col8a1 wildtype control cells (FIG. 8). Anti-VEGF treatment suppressed migration and proliferation of Col8a1 wildtype hESC-derived vECS, however Col8a1 knockout cells showed no response to anti-VEGF (B20) treatment (FIG. 9).

### Example 5: Mouse Model of Angiogenesis

Col8a1 wildtype and Col8a1 knockout mice are injected with 500 μL of growth factor reduced Matrigel (Corning), alone or mixed with 10 ng of recombinant VEGF to the interscapular region under isoflurane anesthesia. Ten days after implantation, mice are euthanized, and Matrigel plugs are collected for cryosection and neovascularization quantification.

### Example 6: Mouse Model of Kidney Injury

Col8a1 wildtype and Col8a1 knockout mice are subject to unilateral renal ischemia-reperfusion injury using the following procedure. Following induction of a surgical plane of anesthesia, both flanks of each mouse (from the scapular area to the pelvic area) are shaved and prepped with alcohol, betadine, and alcohol.

The mouse is placed on a heating pad laying on the left side. A surgical depth of anesthesia is confirmed by absence of reflexes/toe pinch. Once depth of anesthesia is confirmed, a 1 cm incision through the skin and muscle is made on the right flank along the back to expose the right kidney. The renal pedicle is isolated for ligation with a 3-0 silk suture. After ligation, the right kidney is removed. The left kidney is then be exposed in the same way with an incision on the left flank. The renal pedicle is isolated for clamping with a micro-aneurysm clip. After unilateral clamping of the renal pedicles to effect ischemia for 40-60 minutes, the clamps are removed to allow reperfusion. The muscle layer is closed with 4-0 Vicryl suture and skin incisions are closed with wound clips.

For the sham operation, an incision in the skin and muscle layer is made without removal of the kidney. The muscle layer is closed with 4-0 Vicryl suture and skin incisions will be closed with wound clips.

Immediately after the wound closure 0.5 mL warm sterile lactated Ringer's solution, USP is injected intraperitoneally to each mouse to assist with recovery. The animal is kept on a heating pad until it fully recovers from anesthesia before being returned to its cage.

Wound healing at the kidney injury site is assessed for both Col8a1 wildtype and Col8a1 knockout mice.

### Example 7: Mouse Model of Lung Injury

Pulmonary fibrosis is characterized by chronic lung inflammation and abnormal tissue repair, leading to replacement of normal functional structures with an abnormal accumulation of fibroblasts and deposition of collagen in the interstitium and alveolar spaces. Bleomycin-induced lung fibrosis in rodents is widely used by many laboratories and reproduces many typical features of human idiopathic lung fibrosis (IPF). Bleomycin induces double stranded DNA breaks caused by the release of free radicals. This leads to the death of alveolar epithelial cells, inflammation (neutrophil recruitment and cytokine production), tissue repair, and fibrosis in the lung.

Col8a1 wildtype and Col8a1 knockout mice are subject to bleomycin injury to the lung. Wound healing at the injury site is assessed and compared in both Col8a1 wildtype and Col8a1 knockout mice.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will be apparent to those skilled in the art upon review of this specification and the below-listed claims. The full scope of the disclosure should be determined by reference to the claims, along with their full scope of equivalents, and the specification, along with such variations.

1. A method for determining the efficacy of an agent in modulating angiogenesis in a tissue comprising the steps of:
(a) inducing neovascularization in the tissue of a control subject and a test subject, wherein the Collagen Type VIII Alpha 1 Chain (Col8a1) gene has been knocked out or expression of the Col8a1 gene has been knocked down in both the control and test subjects;
(b) administering the agent to the test subject; and
(c) measuring the size of one or more lesions resulting from the neovascularization in the control and test subjects;
wherein the agent modulates angiogenesis in the tissue if the size of the one or more lesions in the test subject is smaller than the size of the one or more lesions in the control subject.

2. The method of claim 1, wherein the tissue is retina.

3. The method of claim 2, wherein the neovascularization is induced by application of a laser to the basement membrane of an eye of the control subject and the test subject.

4. The method of claim 1, wherein the tissue is a tumor.

5. The method of claim 4, wherein the neovascularization is induced by transplantation of a cell matrix plug.

6. The method of any one of claims 1-5, wherein the agent is administered to the test subject after the induction of neovascularization.

7. The method of any one of claims 1-5, wherein the agent is administered to the test subject before the induction of neovascularization.

8. The method of any one of claims 1-5, wherein the agent is administered to the test subject during the induction of the neovascularization.

9. A method for determining the efficacy of an agent in promoting wound healing or inhibiting fibrosis of an injured tissue, comprising the steps of:
(a) inducing one or more lesions by injuring the tissue of a control subject and a test subject, wherein the Collagen Type VIII Alpha 1 Chain (Col8a1) gene has been knocked out or expression of the Col8a1 gene has been knocked down in both the control and test subjects;
(b) administering the agent to the test subject; and
(c) measuring the size of the one or more lesions and/or one or more fibrotic scars formed at the one or more lesions in the control and test subjects;
wherein the agent promotes wound healing in the injured tissue if the size of the one or more lesions in the test subject is smaller than the size of the one or more lesions in the control subject, and/or if the size of the one or more fibrotic scars in the test subject is smaller than the size of the one or more fibrotic scars in the test subject; and wherein the agent inhibits fibrosis of the injured tissue if the size of the one or more fibrotic scars in the test subject is smaller than the size of the one or more fibrotic scars in the test subject.

10. The method of claim 9, wherein the injured tissue is skin or kidney.

11. The method of claim 10, wherein the tissue injury is caused by surgical incision or skin biopsy punch.

12. The method of claim 9, wherein the injured tissue is retina.

13. The method of any one of claims 9-12, wherein the agent is administered to the test subject after the one or more lesions are induced.

14. The method of method of any one of claims 9-12, wherein the agent is administered to the test subject before the one or more lesions are induced.

15. The method of any one of claims 9-12, wherein the agent is administered to the test subject during the induction of the one or more lesions.

16. A method for determining the efficacy of an agent in treating or preventing age-related macular degeneration (AMD) in a subject comprising the steps of:
(a) inducing one or more lesions in retina of a control subject and a test subject, wherein the Collagen Type VIII Alpha 1 Chain (Col8a1) gene has been knocked out or expression of the Col8a1 gene has been knocked down in both the control and test subjects;
(b) administering the agent to the test subject; and
(c) measuring the size of the one or more lesions in the control and test subjects;
wherein the agent treats or prevents AMD if the size of the one or more lesions in the test subject is smaller than the size of the one or more lesions in the control subject.

17. The method of claim 16, wherein the AMD is neovascular AMD.

18. The method of claim 16, wherein the one or more lesions are induced by application of a laser to the basement membrane of an eye of the control subject and the test subject.

19. The method of claim 18, wherein the application of the laser causes choroidal neovascularization.

20. The method of claim 16, wherein the AMD is geographic atrophy (GA).

21. The method of claim 16, wherein the one or more lesions are induced by injection of sodium iodate into the control subject and the test subject.

22. The method of claim 21, wherein the sodium iodate is administered intravitreally or retro-orbitally.

23. The method of claim 21, where in the sodium iodate is administered systemically.

24. The method of claim 23, wherein the sodium iodate is administered intravenously.

25. The method of any one of claims 16-24, wherein the agent is administered to the test subject after the one or more lesions are induced.

26. The method of any one of claims 16-24, wherein the agent is administered to the test subject before the one or more lesions are induced.

27. The method of any one of claims 16-24, wherein the agent is administered to the test subject during the induction of the one or more lesions.

28. The method of any one of claims 16-27, wherein the lesion size is measured between 5 and 10 days after the one or more lesions are induced.

29. The method of claim 28, wherein the lesion size is measured 7 days after the one or more lesions are induced.

30. The method of any one of claims 1-29, wherein the size of the one or more lesions or fibrotic scars is measured by fluorescent, histological and/or optical coherence tomography analysis.

31. The method of claim 30, wherein the size of the one or more lesions or fibrotic scars is measured by fluorescent microscopy.

32. The method of any one of claims 16-31, wherein the agent is administered to the test subject intravitreally.

33. The method of claim 32, wherein the agent is administered to the test subject by intravitreal injection or through an intravitreal device.

34. A method for determining the efficacy of an agent in treating a tumor in a subject comprising the steps of:
(a) inducing tumor formation in a control subject and a test subject, wherein the Col8a1 gene has been knocked out or expression of the Col8a1 gene has been knocked down in both the control and test subjects;
(b) administering the agent to the test subject; and
(c) measuring the number of tumors formed and/or the size of one or more tumors in the control and test subjects;
wherein the agent treats the tumor if the number of tumors in the test subject is fewer than the number of tumors in the control subject and/or the average size of the one or more tumors measured in the test subject is smaller than the average size of the one or more tumors measured in the control subject.

35. The method of claim 34, wherein the agent is administered to the test subject after the tumor formation of one or more tumors in the test subject.

36. The method of claim 34, wherein the agent is administered to the test subject before the formation of one or more tumors in the test subject.

37. The method of claim 34, wherein the agent is administered to the test subject during the induction of tumor formation.

38. The method of any one of claims 341-37, wherein tumor formation is induced by xenograft.

39. The method of claim 38, wherein tumor formation is induced by subcutaneous injection of cells from a tumor cell line.

40. The method of claim 39, wherein tumor number and size are evaluated between 2 and 21 days after injection.

41. The method of claim 40, wherein tumor number and size are evaluated 10 days after injection.

42. The method of any one of claims 1-41, wherein the control subject and the test subject are mammals.

43. The method of claim 42, wherein the control subject and the test subject are rodents, such as mice.

44. The method of claim 42, wherein the control subject and the test subject are non-human primates.

45. The method of any one of claims 1-31 and 34-44, wherein the agent is administered to the test subject systemically or locally.

46. The method of any one of claims 1-31 and 34-44, wherein the agent is administered to the test subject intravitreally, intravenously, intraperitoneally, orally, subcutaneously, or intramuscularly.

47. The method of any one of claims 1-46, wherein the subject is resistant or refractory to treatment with a VEGF inhibitor.

48. The method of any one of claims 1-47, further comprising administering to the control subject and the test subject a VEGF inhibitor.

49. The method of claim 48, wherein the VEGF inhibitor is administered at least three times.

50. The method of claim 48 or 49, wherein the VEGF inhibitor is administered prior to administration of the agent or simultaneously as administration of the agent.

51. The method of any one of claims 47-50, wherein the VEGF inhibitor is an anti-VEGF antibody.

52. A method for determining the efficacy of an agent in modulating angiogenesis or promoting wound healing in injured cells comprising the steps of:
(a) injuring a control culture of cells and a test culture of cells, wherein the Collagen Type VIII Alpha 1 Chain (Col8a1) gene in the cells has been knocked out or expression of the Col8a1 gene has been knocked down in both the control and test cell cultures;
(b) contacting the cells of the test culture of cells with the agent; and
(c) subsequently evaluating migration of cells across the injury and/or cell proliferation in the control culture of cells and the test culture of cells;
wherein the agent is effective in modulating angiogenesis if there is a difference in migration of cells across the injury and/or cell proliferation between the test culture of cells and the control culture of cells, or
wherein the agent is effective in promoting wound healing if the migration of cells across the injury and/or cell proliferation is faster in the test culture of cells than in the control culture of cells.

53. The method of claim 52, wherein the method is for determining the efficacy of an agent in promoting angiogenesis in injured cells.

54. The method of claim 52, wherein the control and test cells are endothelial cells, fibroblasts or retinal pigment epithelial (RPE) cells.

55. The method of claim 52, wherein the control and test cells are vascular endothelial cells (vECs).

56. The method of any one of claims 52-55, wherein the cells are human cells.

57. The method of any one of claims 52-56, wherein the injuring is a scratch.

58. The method of any one of claims 52-57, wherein the contacting step comprises adding the agent to the culture medium of the test culture of cells after the test culture of cells is injured.

59. The method of any one of claims 52-57, wherein the contacting step comprises adding the agent to the culture medium of the test culture of cells before the test culture of cells is injured.

60. The method of any one of claims 1-59, wherein the Col8a1 gene is knocked out using gene editing technologies.

61. The method of claim 60, wherein the gene editing technology is CRISPR/Cas9.

62. The method of any one of claims 1-61, wherein the agent is selected from the group consisting of a small molecule, an antibody, a polypeptide, a polynucleotide, and a gene therapy.

63. The method of any one of claims 1-62, wherein the agent is not a VEGF inhibitor.

64. The method of any one of claims 1-63, wherein the subject or cells have wildtype Collagen Type VIII Alpha 2 Chain (Col8a2) gene.


SEQUENCE LISTING (11 sequences)

SEQ ID NO: 1 - RNA, 20 nt, synthetic construct
  source 1..20: mol_type = other RNA
gtaggagctacgtcaaaatt

SEQ ID NO: 2 - RNA, 20 nt, synthetic construct
  source 1..20: mol_type = other RNA
cagttaagagtgttcggcac

SEQ ID NO: 3 - DNA, 21 bp, synthetic construct
  source 1..21: mol_type = other DNA
ttgtactagatgctccttcct

SEQ ID NO: 4 - DNA, 18 bp, synthetic construct
  source 1..18: mol_type = other DNA
gcttcctgatggtttggt

SEQ ID NO: 5 - DNA, 24 bp, synthetic construct
  source 1..24: mol_type = other DNA
ttggtattgctgttacacttattg

SEQ ID NO: 6 - protein, 744 aa, Mus musculus
MAVPPRPLQLLGILFIISLNSVRLIQAGAYYGIKPLPPQIPPQIPPQIPQYQPLGQQVPHMPLGKDGLSMGKEMPHMQYGKEYPHLPQYMKEIPPVPRMGKEVVPKKGKGEVPLASLRGEQGPRGEPGPRGPPGPPGLPGHGMPGIKGKPGPQGYPGIGKPGMPGMPGKPGAMGMPGAKGEIGPKGEIGPMGIPGPQGPPGPHGLPGIGKPGGPGLPGQPGAKGERGPKGPPGPPGLQGPKGEKGFGMPGLPGLKGPPGMHGPPGPVGLPGVGKPGVTGFPGPQGPLGKPGPPGEPGPQGLIGVPGVQGPPGMPGVGKPGQDGIPGQPGFPGGKGEQGLPGLPGPPGLPGVGKPGFPGPKGDRGIGGVPGVLGPRGEKGPIGAPGMGGPPGEPGLPGIPGPMGPPGAIGFPGPKGEGGVVGPQGPPGPKGEPGLQGFPGKPGFLGEVGPPGMRGLPGPIGPKGEGGHKGLPGLPGVPGLLGPKGEPGIPGDQGLQGPPGIPGIVGPSGPIGPPGIPGPKGEPGLPGPPGFPGVGKPGVAGLHGPPGKPGALGPQGQPGLPGPPGPPGPPGPPAVMPTPSPQGEYLPDMGLGIDGVKPPHAYAGKKGKHGGPAYEMPAFTAELTVPFPPVGAPVKFDKLLYNGRQNYNPQTGIFTCEVPGVYYFAYHVHCKGGNVWVALFKNNEPMMYTYDEYKKGFLDQASGSAVLLLRPGDQVFLQMPSEQAAGLYAGQYVHSSFSGYLLYPM

SEQ ID NO: 7 - protein, 723 aa, synthetic construct
MAVPPRPLQLLGILFIISLNSVRLIQAGAYYGIKPLPPQIPPQIPPQIPQYQPLGQQVPHMPLGKDGLSMGKEMPHMQYGKEYPHLPQYMKEIPPVPRMGKEVVPKKGKGPPGPPGLPGHGMPGIKGKPGPQGYPGIGKPGMPGMPGKPGAMGMPGAKGEIGPKGEIGPMGIPGPQGPPGPHGLPGIGKPGGPGLPGQPGAKGERGPKGPPGPPGLQGPKGEKGFGMPGLPGLKGPPGMHGPPGPVGLPGVGKPGVTGFPGPQGPLGKPGPPGEPGPQGLIGVPGVQGPPGMPGVGKPGQDGIPGQPGFPGGKGEQGLPGLPGPPGLPGVGKPGFPGPKGDRGIGGVPGVLGPRGEKGPIGAPGMGGPPGEPGLPGIPGPMGPPGAIGFPGPKGEGGVVGPQGPPGPKGEPGLQGFPGKPGFLGEVGPPGMRGLPGPIGPKGEGGHKGLPGLPGVPGLLGPKGEPGIPGDQGLQGPPGIPGIVGPSGPIGPPGIPGPKGEPGLPGPPGFPGVGKPGVAGLHGPPGKPGALGPQGQPGLPGPPGPPGPPGPPAVMPTPSPQGEYLPDMGLGIDGVKPPHAYAGKKGKHGGPAYEMPAFTAELTVPFPPVGAPVKFDKLLYNGRQNYNPQTGIFTCEVPGVYYFAYHVHCKGGNVWVALFKNNEPMMYTYDEYKKGFLDQASGSAVLLLRPGDQVFLQMPSEQAAGLYAGQYVHSSFSGYLLYPM

SEQ ID NO: 8 - RNA, 20 nt, synthetic construct
  source 1..20: mol_type = other RNA
gagttccatcaggctcatcc

SEQ ID NO: 9 - RNA, 20 nt, synthetic construct
  source 1..20: mol_type = other RNA
ttgcgaactaagtattagac

SEQ ID NO: 10 - DNA, 20 bp, synthetic construct
  source 1..20: mol_type = other DNA
ggttcctcccacaggtgatg

SEQ ID NO: 11 - DNA, 27 bp, synthetic construct
  source 1..27: mol_type = other DNA
tgtcacactgaggggcattttgttggt